(19) United States
(12) Patent Application Publication
(10) Pub. No.: US 2023/0295549 A1
(43) Pub. Date: Sep. 21, 2023

(54) METHODS, ASSEMBLY AND SYSTEM FOR HIGH-THROUGHPUT, PARALLEL MONITORING OF REACTION SYSTEMS

(71) Applicant: Danmarks Tekniske Universitet, Kongens Lyngby (DK)

(72) Inventors: Simo Abdessamad Baallal Jacobsen, Kongens Lyngby (DK); Suresh Sudarsan, Kongens Lyngby (DK)

(21) Appl. No.: 18/041,455

(22) PCT Filed: Aug. 20, 2021
(86) PCT No.: PCT/EP2021/073150

§ 371 Date: Feb. 13, 2023

(30) Foreign Application Priority Data
Aug. 20, 2020 (EP) 20191858.8

(51) Int. Cl. C12M 3/00; G01N 21/80; G01N 21/82; C12M 1/00

(52) U.S. Cl. CPC C12M 23/42; G01N 21/80; G01N 21/82; C12M 23/48; C12M 23/22; G01N 2021/7786

(57) ABSTRACT

The present invention relates assembly and a system for improved, high-throughput cost-effective cell culture, cultivations, fermentations and assays. Taking advantage of an assembly which can adopt two different configurations, efficient methods for monitoring a plurality of reaction systems and/or for cultivating and/or screening cell populations are provided.

### TECHNICAL FIELD

The present invention relates to an assembly and a system for improved, high-throughput cost-effective cell culture, cultivations, fermentations and assays. Taking advantage of an assembly which can adopt two different configurations, efficient methods for monitoring a plurality of reaction systems and/or for cultivating and/or screening cell populations are provided.

### BACKGROUND

The constant search for new biopharmaceutical drugs against rising incidence of cancer, infectious diseases and pandemics, rising genetic disorders, as well as the ever increasing interest for e.g. genomics and proteomics, growing awareness about the benefits of cell culture-based vaccines, increasing interest on personalised medicine and in sustainable microbial cell factories, has for several years driven the growth of cell culture, biopharmaceutical, bioprocessing and fermentation markets.

However, working with cell culture and microbial cultivations still suffers from limitations and challenges. The major challenge, that hampers efforts to ramp up developments and research when working with cell culture and cultivations, is the huge bottleneck of the low throughput and the high costs associated with existing systems.

WO 2020/079211 describes an electrophoresis assembly comprising two or more frames for holding a separation matrix such as a gel cassette, where each frame comprises at least one inner frame, and wherein the two or more frames are attached together such that in a first configuration the two or more frames abut each other in a longitudinal direction, and in a second configuration the two or more frames are in extension of each other. The electrophoresis assembly is not suitable for holding a plurality of containers such as cell cultivation systems.

U.S. Pat. No. 7,422,893 discloses a device for growing cells under static or variable pressure and comprises a pressure adaptor consisting of two lateral frames joined by a hinge, between which a cell cassette can be held in place. The cell cassette comprises of two opposite and gas-permeable membrane sides and can be used to grow cells in 2D. The cell cassette does not comprise a plurality of containers holding reaction systems.

Therefore, there is a strong need for improved assemblies and systems for high-throughput cost-effective cell culture, cultivations and fermentations.

### SUMMARY

Herein are provided an assembly and a system for improved, high-throughput cost-effective cell culture, cultivations, fermentations and assays. Taking advantage of an assembly which can adopt two different configurations, efficient methods for monitoring a plurality of reaction systems and/or for cultivating and/or screening cell populations are provided. These methods allow optical measurements from a lateral side of the assembly, as described herein in detail. The assembly and system can be configured to match SBS formats or SLAS standards, which allows for efficient handling by enabling the use of automated liquid handlers, colony pickers, replicators and the like.

The present assemblies and systems can easily be scaled up either by increasing the number of containers, and hence reaction systems, that they can accommodate, or by increasing their dimensions, particularly their vertical dimensions. The present assemblies and systems can thus be used for a wide range of applications, including monitoring and screening of a plurality of liquid compositions and/or reaction mixtures which can be provided in a wide range of different volumes, from micro-systems, and even nano-systems, to macro-systems.

Herein is thus provided an assembly (1) comprising two or more frames (2, 2′) and two or more cassettes (3, 3′), wherein each cassette comprises a plurality of containers or is configured to hold a plurality of containers (4, 4′) in a vertical direction,
wherein each frame is configured to receive at least one cassette,
wherein each frame comprises an opening in an upper end
and wherein:
   a) each frame further comprises a hinge (5) or an internal slidable connection, wherein the two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
   b) the assembly comprises a further slidable connection (12, 12′), wherein the two or more frames are releasably connected to one another via said further slidable connection,
      such that in a first configuration the two or more frames abut each other in a longitudinal direction, and in a second configuration the two or more frames do not abut each other in a longitudinal direction while still connected to one another via said further slidable connection,
and wherein each frame and cassette are configured to allow optical access to the containers from the side of said each frame and cassette at least when the assembly is in the second configuration.

Also provided is a system comprising:
i) an assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end and wherein:
      a) each frame further comprises a hinge (5) or an internal slidable connection, wherein the at two or more frames are pivotably hinged to one another via said hinge or wherein the two or more frames are connected to one another via said internal slidable connection,
   such that in a first configuration the two or more frames abut each other in a longitudinal direction, and in a second configuration the two or more frames are in extension of each other, and/or
      b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
and wherein each frame and cassette are configured to allow optical access to the containers from the side of said each frame and cassette at least when the assembly is in the second configuration,
ii) one or more detection means comprising at least one optical detection means (14, 14′); and
iii) optionally, analysis means.

Also provided is a method for monitoring a plurality of reaction systems, said method comprising the steps of:
i) providing a system as disclosed herein or an assembly (1) comprising two or more frames (2, 2′) and two or more cassettes (3, 3′), wherein each cassette comprises a plurality of containers or is configured to hold a plurality of containers (4, 4′) in a vertical direction, wherein each frame is configured to receive at least one cassette, wherein each frame comprises an opening in an upper end and wherein:
a) each frame further comprises a hinge (5) or an internal slidable connection, wherein the at two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
      and wherein each frame and cassette are configured to allow optical access to the containers from the side of said each frame and cassette at least when the assembly is in the second configuration,
      wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical detection means as defined herein, thereby monitoring the plurality of reaction systems.

Also provided is a method for cultivating and/or screening a plurality of cells, said method comprising the steps of:
i) providing a system as disclosed herein or an assembly (1) comprising two or more frames (2, 2′) and two or more cassettes (3, 3′), wherein each cassette comprises a plurality of containers or is configured to hold a plurality of containers (4, 4′) in a vertical direction, wherein each frame is configured to receive at least one cassette, wherein each frame comprises an opening in an upper end and wherein:
a) each frame further comprises a hinge (5) or an internal slidable connection, wherein the two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration,
      wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical sensors as defined herein,
   the method further comprising introducing said cells in the containers before or together with step iii) or iv), thereby cultivating and/or screening the plurality of cells.

### DESCRIPTION OF DRAWINGS

FIG. 1 shows an embodiment of an assembly (1), comprising frames (2, 2′) each configured to receive one cassette (3, 3′). The cassettes each comprise a plurality of containers (4, 4′), here capped by a capping strip (10) and comprising an opening in their lower end, which is sealed by plugs (8). The containers here have three optical sensors (11, 11′, 11″). The frame further comprises a row of needles (9) which can penetrate the plugs (8). The frames are here connected to one another via hinges (5).

FIG. 2 shows another embodiment of an assembly (1), comprising frames (2, 2′) each configured to receive one cassette (3, 3′). The cassettes each comprise a plurality of containers (4, 4′), here capped by a capping strip (10) and comprising an opening in their lower end, which is sealed by plugs (8). The containers here have three optical sensors (11, 11′, 11″). The frame further comprises a row of needles (9) which can penetrate the plugs (8). The frames are here connected to one another via a further slidable connection, here in the form of a cylinder (12).

FIG. 3 shows details of a single frame (2) and cassette (3), in exploded view in FIG. 3A. The containers (4, 4′), capped by a capping strip (10) and further comprising an opening in their lower end, sealed by individual plugs (8), have on their lateral surface three optical sensors (11, 11′, 11″), and can be inserted in the frame from an upper end, thanks to ridges and grooves on the sides of the cassette and frame, respectively. The frame further comprises a row of needles (9) which can penetrate the plugs (8). FIG. 3B shows the containers inserted in the cassette, which in turn is shown inserted in the frame.

FIG. 4 shows the assembly of FIG. 1, here in an intermediate configuration. The frames here comprise magnets (15, 15′), which help maintain the assembly in a first configuration.

FIG. 5 shows details of a single cassette (3) configured to hold a first row (6) and a second row (6′), each row with 24 containers. The two rows are separated by a non-transparent barrier (7). The containers (4, 4′) are capped by a capping strip (10) and further comprise an opening in their lower end, sealed by individual plugs (8), and have on their lateral surface three optical sensors (11, 11′, 11″).

FIG. 6 shows an example of an assembly (1), here placed in an incubator chamber (13) comprising two detections means for the non-invasive optical sensor spots (14, 14′).

FIG. 7 shows an example of an assembly (1), here placed in an incubator chamber (13) comprising one detections means for the non-invasive optical sensor spots (14). The assembly in the second configuration here forms a closed perimeter.

FIG. 8 shows an assembly of FIG. 2, in a second configuration. Detection is possible for a single frame and cassette, to which two detections means for the non-invasive optical sensor spots (14, 14′) have optical access from the side. FIG. 8A shows the assembly ready for optical measurements to be performed. FIG. 8B shows the assembly while optical measurements by the detection means (14, 14′) are ongoing.

FIG. 9 illustrates how the present assemblies, here an assembly of FIG. 2, can easily be scaled up by increasing a vertical dimension of the assembly. The assembly of FIG. 9A can be scaled up vertically as shown in FIGS. 9B and 9C.

### DETAILED DESCRIPTION

Assembly

The present disclosure relates to an assembly which is particularly useful for monitoring a plurality of reaction systems. For example, the assembly can be used to perform micro or mini cultivation systems, for example micro- or mini-fermentations, and can inter alia be used to propagate and/or screen a library of candidate cells for production of compounds of interest, for example, but not only, for production of new drugs or antibiotics. For example, the reaction system may be a cellular reaction system, comprising a reaction system in which cells are present and in which some chemical reactions occur or are expected to occur, or a catalytic reaction system, comprising for example enzymes such as purified enzymes or cellular extracts comprising active enzymes, in which enzymatic reactions occur. Another example of a reaction system is a catalytic reaction system comprising one or more catalysts and substrates, in which chemical reactions occur or are expected to occur.

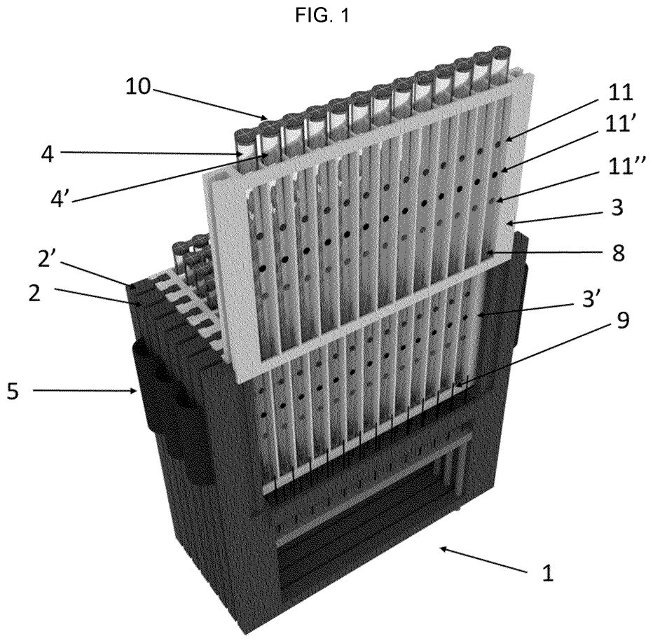

The present assembly, some embodiments of which are shown in the FIGS. 1), is particularly amenable for high-throughput methods. The assembly can be arranged in several configurations. In a first, closed configuration, the assembly is present in a compact form, which matches SBS formats or SLAS standards. This facilitates preparation of the reaction systems to be held in the assembly, as it allows the use of high-throughput devices, for example automated, high-throughput devices, thus enabling simultaneous handling or sampling of multiple reaction systems. In a second, open configuration, the assembly is at least partly open or unfolded to allow access to the reaction systems, thereby enabling monitoring of the reaction systems in a precise manner. The assembly can also adopt a number of intermediate configurations.

The assembly comprises two or more frames (2, 2′) and two or more cassettes (3, 3′), wherein each cassette comprises a plurality of containers or is configured to hold a plurality of containers (4, 4′) in a vertical direction,
wherein each frame is configured to receive at least one cassette,
wherein each frame comprises an opening in an upper end
and wherein:
   a) each frame further comprises a hinge (5) or an internal slidable connection, wherein the two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
   b) the assembly comprises a further slidable connection (12, 12′), wherein the two or more frames are releasably connected to one another via said further slidable connection,
      such that in a first configuration the two or more frames abut each other in a longitudinal direction, and in a second configuration the two or more frames do not abut each other in a longitudinal direction while still connected to one another via said further slidable connection,
         and wherein each frame and cassette are configured to allow optical access to the containers from the side of said each frame and cassette at least when the assembly is in the second configuration.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,
wherein each frame is configured to receive at least one cassette,
wherein each frame comprises an opening in an upper end
and wherein each frame further comprises a hinge (5) or an internal slidable connection, wherein the two or more frames are pivotably hinged to one another via said hinge or wherein the two or more frames are connected to one another via said internal slidable connection,
such that in a first configuration the two or more frames abut each other in a longitudinal direction, and in a second configuration the two or more frames are in extension of each other,
   and wherein each frame and cassette are configured to allow optical access to the containers from the side of said each frame and cassette at least when the assembly is in the second configuration.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,
wherein each frame is configured to receive at least one cassette,
wherein each frame comprises an opening in an upper end,
and wherein the assembly comprises a further slidable connection (12, 12′),
wherein the two or more frames are releasably connected to one another via said further slidable connection,
such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,
wherein each frame is configured to receive at least one cassette,
wherein each frame comprises an opening in an upper end,
and wherein each frame further comprises a hinge (5) or an internal slidable connection, wherein the two or more frames are pivotably hinged to one another via said hinge or wherein the two or more frames are connected to one another via said internal slidable connection,
such that in a first configuration the two or more frames abut each other in a longitudinal direction, and in a second configuration the two or more frames are in extension of each other,
and wherein the assembly comprises a further slidable connection (12, 12′), wherein the two or more frames are releasably connected to one another via said further slidable connection,
such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration.

The containers being optically accessible from the side of said each frame and cassette at least when the assembly is in the second configuration means that the assembly is optically permeable to electromagnetic radiation, in particular visible light, infrared light and/or ultraviolet light. In other words, electromagnetic radiation, in particular light, can enter the assembly from the side at least when it is in the second configuration, and can reach the containers. Preferably, optical access is possible to the inner part of the containers, so that optical access to the reaction systems contained therein is possible when the assembly is in use. In other words, electromagnetic radiation, in particular visible light, infrared light and/or ultraviolet light, preferably can reach the inside of the containers and reach the reaction systems contained therein at least when the assembly is in use.

The electromagnetic radiation may be a UV radiation, i.e. having a wavelength in the range of 10 nm to 400 nm, or it may be visible light, i.e. having a wavelength in the range of 380 nm to 750 nm, or it may be infrared radiation, i.e. having a wavelength in the range of 700 nm to 1 mm.

The assemblies of the present disclosure may be reusable or may be intended for a single use. Importantly, the assemblies of the present disclosure are not configured to hold a separation matrix such as a gel or a membrane.

Cassettes

The assembly comprises two or more cassettes, which are exemplified in the figures as 3 and 3′. Each cassette either comprises a plurality of containers, or is configured to hold a plurality of containers (4, 4′) in a vertical direction. It will be understood that the containers may be external to the cassettes, as shown in the figures, and that they can be introduced in the cassettes in a vertical direction. In such embodiments, the cassettes comprise an opening at least in an upper end to allow introduction of the containers therein. The cassettes may be equipped with locking means allowing the containers to remain in place even when the assembly is in use and mechanical stresses, for example stirring, are applied to it.

The containers may however, in alternative embodiments, be integrated in the cassettes, which may be formed for example by mould injection in such a way that chambers are provided therein, which can act as containers for reaction systems as described herein. In such cases, the containers are thus an integral part of the cassettes.

The cassettes are made of a material such that optical access to the containers held or comprised therein is possible, at least when the assembly is in a second configuration. The cassettes allow light transmission for proper monitoring of the containers and the reaction systems. Thus in some embodiments where the containers are external to the cassettes, the cassettes are hollow or comprise a cavity allowing optical access to the containers, and are further shaped to maintain the containers in place; this can be achieved as is known in the art, e.g. the cassettes may be manufactured by mold injection. In other embodiments, the containers are internal to the cassettes, which then comprise a window made of a material allowing light transmission, such as a transparent material, allowing optical access to the containers, i.e. the inner volume comprised in the containers. In embodiments where the cassettes comprise a window made of a material allowing light transmission, the skilled person will have no difficulty in determining which material to use depending on which light should access the inner volume comprised in the containers. The light may be ultraviolet light, infrared light or visible light.

Preferably, the cassettes are made of a material which is structurally stable under the conditions to be used when monitoring the reaction systems. This is particularly important when the containers are an integral part of the cassettes. Preferably, the material from which the cassettes are manufactured is inert, and/or does not oxidise when contacted with fluids such as culture medium or buffers, and/or does not rust easily. Materials suitable for manufacturing such assemblies are known to the skilled person.

In order to allow optical access to the reaction systems within the cassettes, the two or more cassettes may comprise an opening on a lateral surface, or a lateral surface or a lateral side of the two or more cassettes may comprise a transparent surface, which allows optical access to the containers held in the assembly at least in the second configuration. Such transparent surface when present is made of a material which allows optical access, in particular transmission of light, for example light of different wavelengths, such as UV light, laser light or blue light, as detailed above.

The cassettes are preferably of a rectangular or square shape. The dimensions of the cassettes are preferably such that containers can be introduced therein, and maintained therein in such a manner that the containers do not move once inserted. Accordingly, particularly in embodiments where the containers are external to the cassette, the cassette preferably has at least one opening for inserting containers therein, e.g. on the upper side. In embodiments where the cassette is formed in such a way that the containers are integrated therein, the cassette may still comprise an opening at least on the upper side. This can allow for additional features enabling monitoring and/or sampling and/or modification of the reaction conditions in the containers, as detailed herein below.

Preferably, the lower side of the cassette is at least partially closed so that the containers do not simply slide through the cassette. In some embodiments, the lower side of the cassette comprises an opening, which allows for additional features enabling monitoring and/or sampling and/or modification of the reaction conditions in the containers, as detailed herein below. In embodiments where the cassette is formed in such a way that the containers are integrated therein, the cassette may still comprise an opening on the lower side.

Additionally, particularly in embodiments where the containers are external to the cassettes and are inserted therein before performing the methods described herein, the frame may further comprise retention means which help maintaining the containers in place. Such retention means may be in the form of a ring, for example a rubber or silicone ring, or in the form of one or more protrusions, which surround each container when inserted, and help keep it in place, particularly when the assembly is submitted to mechanical forces such as resulting from stirring the assembly.

Frames

The assembly also comprises two or more frames. These are exemplified in the figures as 2 and 2′. The frames are configured to each receive at least one cassette, and comprise an opening in an upper end which allows the cassette to be inserted therein. Accordingly, the two or more frames can receive the at least one cassette from an upper end. In some embodiments, each frame can receive one cassette or more, such as two cassettes.

The frames of the assembly are attached together in such a way that the assembly can

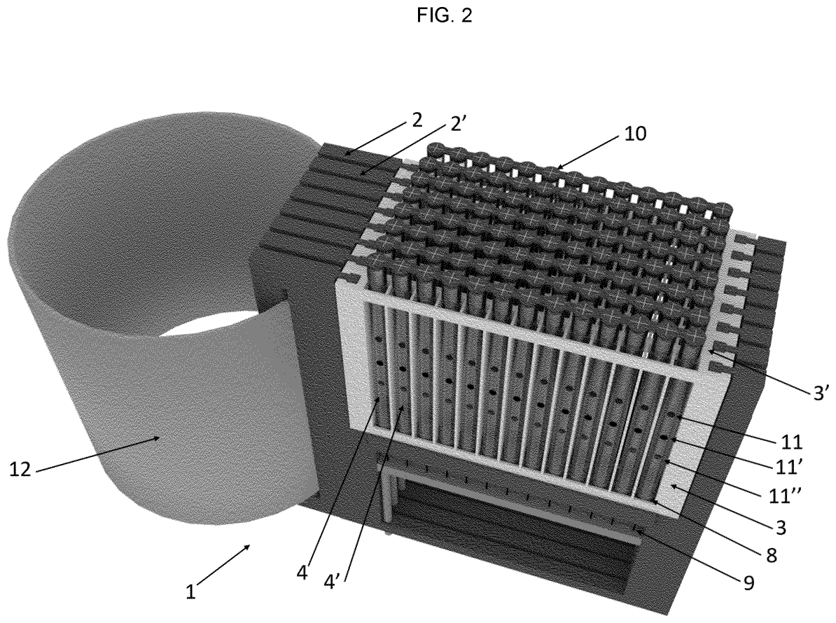

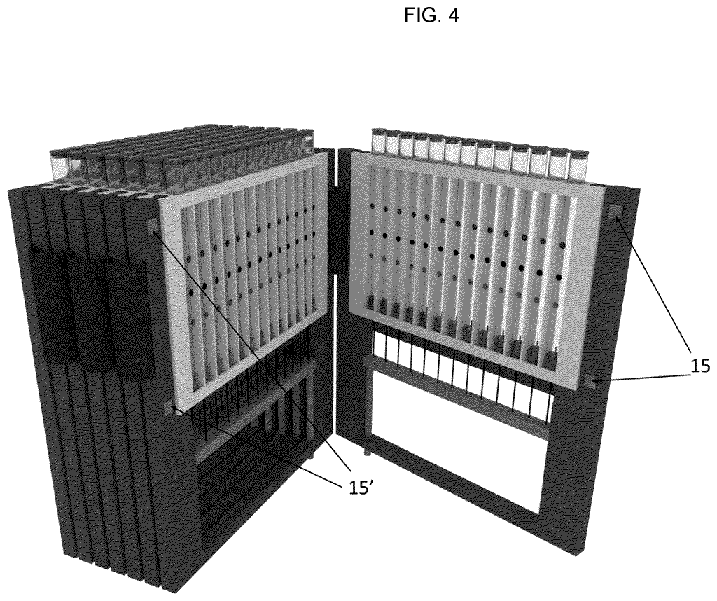

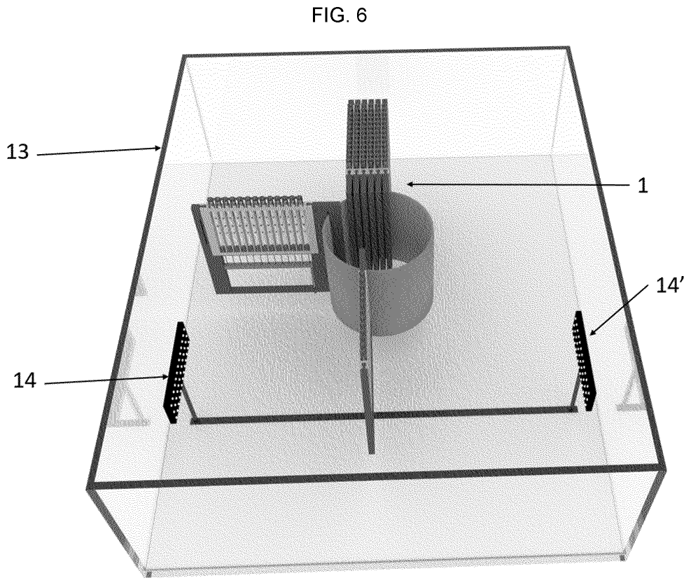

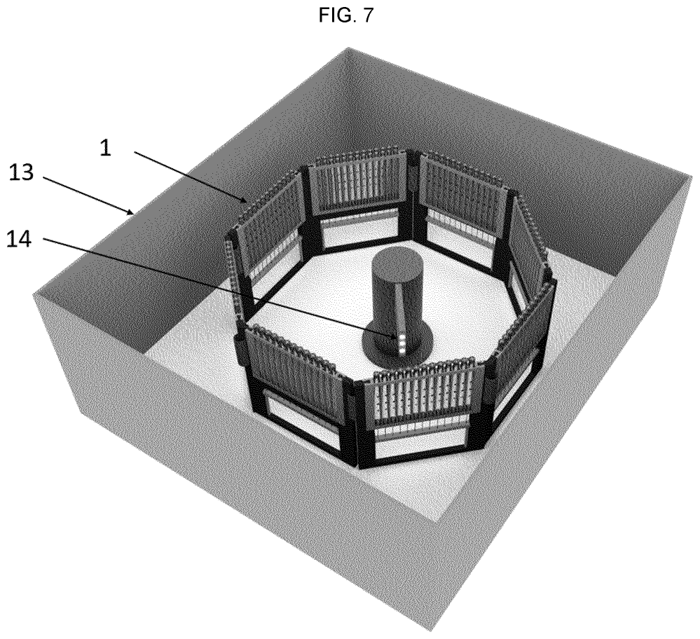

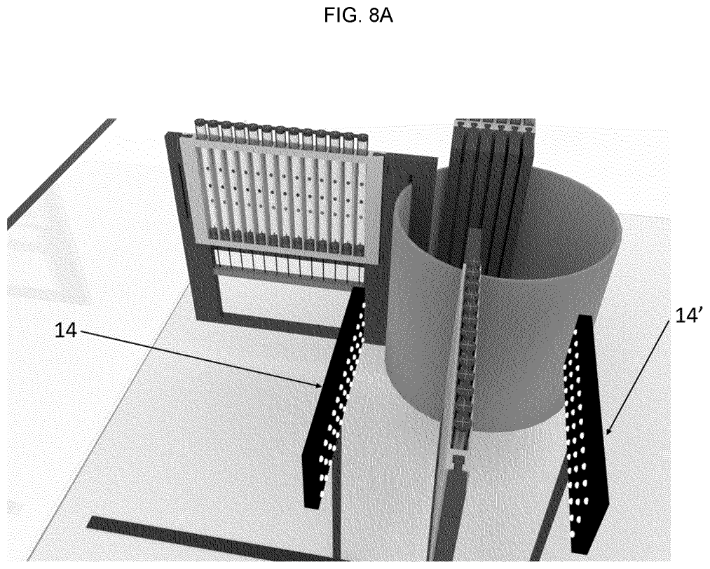

be arranged in different configurations. The frames are thus configured in such a way that they can be arranged in a first configuration (a closed configuration) where they abut each other in a longitudinal direction, as illustrated by way of example on FIGS. 1 and 2. The frames can also adopt a second configuration (an open configuration), which allows optical access to the containers held in the frames from the side, as illustrated by way of example on FIGS. 6 and 7. The frames can also be arranged in intermediate configurations, i.e. any configuration going from the first configuration to the second configuration, as can be seen for example in FIG. 4.

In the first configuration, the two or more frames abut each other in a longitudinal direction, i.e. the biggest lateral side of two adjacent frames are adjacent and parallel to one another along the entire surface of their biggest lateral side. In the first configuration, preferably all frames abut each other, and optical access is, if at all, only possible from one side of the outer frames. In the second configuration, the two or more frames do not abut each other. In the second configuration, not all of the frames abut each other—some frames may still abut each other, but at least one frame is accessible from both sides for optical measurements. Access to the containers from the side of each frame, in particular optical access, is thus possible at least when the assembly is in the second configuration.

In other words, in a first configuration, when each frame of the assembly comprises cassettes comprising containers inserted therein or internal chambers or containers as described herein above, at least one lateral side of the containers may not be directly accessible, in particular optical access to said lateral side may not be possible, as access may be prevented by the containers held or present in the adjacent cassettes or frames. In a second configuration, when each frame of the assembly comprises cassettes comprising containers inserted therein or internal chambers or containers as described herein above, at least one lateral side of the containers is directly accessible, for example optical access is possible. The term “lateral side” herein refers to a side of the containers, usually the biggest lateral side. If the containers are shaped as cylinders, i.e. they have a circular cross-section, the term refers to the lateral wall with the greatest area. If the containers have a square or rectangular cross-section, the term refers to one of the four walls having the greatest area.

The term “abutting each other in a longitudinal direction” in relation to two frames refers herein to the fact that the frames abut each other on their biggest lateral side, i.e. the side that defines the biggest plane of the frame.

In a second configuration, in embodiments where the assembly comprises frames with a hinge or an internal slidable connection, the two or more frames do not abut each other. Preferably, the two or more frames are in extension of each other. This does not mean that the two or more frames necessarily follow a continuous line, but it should be understood that in the second configuration the two or more frames are in contact with one another essentially only via the hinge or the internal connection. The two or more frames may however be at an angle which is not 180°, for example they can be at a 90° angle, or at any other angle provided that they do not abut each other.

In the second configuration, in embodiments where the assembly comprises a further slidable connection, wherein the two or more frames are releasably connected to one another via said further slidable connection, the two or more frames do not abut each other in a longitudinal direction while still connected to one another via said further slidable connection. In such embodiments the two or more frames typically cannot be in extension of each other in the second configuration, particularly where the further slidable connection is a ring on which the frames are connected.

The assembly when in the second configuration can form a closed perimeter. In such embodiments, the assembly comprises at least three frames, and the two frames located in the extremities of the assembly can be connected to one another; the assembly can thus form a closed perimeter. Depending on the number of frames within the assembly, the closed perimeter thus formed can adopt various shapes. An assembly comprising three frames will have an essentially triangular shape. An assembly comprising an even number of frames can have an essentially rectangular shape. An assembly comprising four frames, or a number of frames which is a multiple of four, can have an essentially square shape. An assembly comprising an even number of frames greater than four, in particular an even number which is not a multiple of four, can have an essentially regular shape. Uneven numbers of frames will result in other shapes.

The assembly can however also be used in configurations where the frames when in the second configuration, do not form a closed perimeter. This is the case in particular for embodiments where the assembly comprises a further slidable connection connecting the two or more frames.

In order to allow optical access, the two or more frames may comprise an inner cavity on a lateral surface, or a lateral surface or a lateral side of the two or more frames may comprise a transparent surface, which allows optical access to the cassette(s) held in the assembly at least in the second configuration. Such transparent surface when present is made of a material which allows optical access, in particular transmission of light, such as UV light, laser light or blue light.

In some embodiments, the assembly comprises at least two frames, such as at least three frames, such as at least four frames, such as at least five frames, such as at least six frames, such as at least seven frames, such as at least eight frames, such as at least nine frames, such as at least ten frames, such as at least eleven frames, such as at least twelve frames, or more. Each frame may comprise one cassette or more, such as two cassettes.

Thus in some embodiments, each frame comprises one cassette. For example the assembly comprises two frames and two cassettes, one cassette in each frame, or the assembly comprises three frames and three cassettes, four frames and four cassettes, five frames and five cassettes, six frames and six cassettes, seven frames and seven cassettes, eight frames and eight cassettes, nine frames and nine cassettes, ten frames and ten cassettes, eleven frames and eleven cassettes, or twelve frames and twelve cassettes, or more.

In other embodiments, each frame comprises two cassettes. For example, the assembly comprises two frames and four cassettes, two in each frame, or the assembly comprises three frames and six cassettes, four frames and eight cassettes, five frames and ten cassettes, six frames and twelve cassettes, seven frames and fourteen cassettes, eight frames and sixteen cassettes, nine frames and eighteen cassettes, ten frames and twenty cassettes, eleven frames and twenty-two cassettes, or twelve frames and twenty-four cassettes, or more.

In some embodiments, some frames comprise one cassette and some frames comprise two cassettes.

Within each cassette, the containers may be arranged in one or more rows, such as two rows. It will however generally be most advantageous to arrange the containers so that if a frame comprises two cassettes, then each cassette comprises or is configured to hold a plurality of containers arranged in a single row. If the frame comprises only one cassette, the plurality of containers can be arranged in a single row, or in two rows.

Each frame may comprise cassettes in which the containers are arranged in one or more rows, preferably in one or two rows, such as a first row and a second row.

Hinge, internal slidable connection and further slidable connection In order to enable the assembly to be arranged in a first configuration and in a second configuration, in some embodiments each frame comprises a hinge or an internal slidable connection. FIG. 1 shows an example of an assembly with frames comprising a hinge (5). An internal slidable connection can be for example ridges or magnets, which allow the frames to be moved in a parallel direction. In such embodiments, the two or more frames in the second configuration can be in extension of each other, as illustrated by way of example in FIG. 7.

Thus in some embodiments, the frames comprise a hinge and are connected in pairs to one another via said hinge. The frame defines a rectangle comprising four sides: an upper side (where the cassette can be introduced), a lower side (holding the cassette in place), a right side and a left side. The hinge is preferably located on the right side and/or on the left side of the frame. The hinge is preferably not located on the upper side or on the lower side. The hinge may be one hinge, two hinges, three hinges or more.

Where the frame(s) comprise(s) a hinge on one side, it may be advantageous for the frame to also include additional connection means on another side, for example magnets. The frame may thus comprise a hinge on at least one side, through which it can be connected to another identical frame, and may further comprise additional connection means such as magnets on another side. The additional connection means may be located on the side of the frame opposite to the hinge, and/or they may be located on the upper and/or the lower end of the frame. Such additional connection means may help maintain the cassettes comprised in the assembly in place to facilitate introduction of a liquid composition such as a liquid reaction mixture or medium such as a growth medium in the containers, as they may prevent the creation of gaps between the frames.

Preferably, the additional connection means are magnets. The magnets may be coated with isolating material or cushioning material.

In other embodiments, the frame comprises an internal slidable connection, for example a ridge, a groove or a slit, which allows the frame to be slidably connected to another frame. Preferably, the internal slidable connection is located on the upper and/or on the lower side of the frame. This allows two frames connected via a slidable connection to slide in relation to one another.

Another way of enabling the assembly to be arranged in a first configuration and in a second configuration can be achieved when the assembly comprises a further slidable connection, an example of which (12) is represented on FIG. 2. Such a further slidable connection may be external to the frames, and may be in the form of a ring or a hollow cylinder to which the two or more frames can be attached. The frames can thus be pushed in the first configuration, and can be maintained in this configuration for example via magnets present on the frames, or by other locking means, while the second configuration is achieved for example by pulling the frames so that at least two adjacent frames no longer abut each other in a longitudinal direction, but are only minimally, if at all, in contact with one another. In some cases, the only contact between two consecutive frames in the second configuration is through the further slidable connection.

The first configuration corresponds to a closed configuration. This configuration is particularly advantageous for introducing a liquid composition such as a liquid or a molten reaction mixture or medium such as a growth medium in the containers held within the assembly, for extracting samples from the containers, or for introducing further elements in the containers, for example to replenish the cultivation medium, as will be detailed herein below. The term “molten reaction” refers here to a reaction system which has been fused or liquefied by heat. For example, pieces of solid medium comprising a solidifying agent such as agar may be introduced in the container and may be liquefied by heat prior to analysis. Conversely, warm medium comprising a solidifying agent such as agar may be introduced in the containers in a liquid form, and may be allowed to solidify upon cooling, e.g. to room temperature. This can be desirable in cases where development of colonies at the surface of the solid medium are to be studied.

The second configuration corresponds to an open configuration. This configuration is particularly advantageous for monitoring the containers within the cassettes, in particular reaction systems comprised therein.

Containers

As detailed herein above, the containers may be external containers which can be inserted in the cassettes, or they may be in the form of internal chambers or cavities within the cassettes.

The containers are fluid-tight and are configured to hold a liquid composition such as a liquid or a molten reaction mixture or medium such as a growth medium.

Each container is thus configured to hold a volume of a liquid composition such as a liquid or a molten reaction mixture or medium such as a growth medium of at least 1 μL, such as at least 2.5 μL, such as at least 5 μL, such as at least 10 μL, such as at least 15 μL, such as at least 25 μL, such as at least 50 μL, such as at least 75 μL, such as at least 0.1 mL, such as at least 0.25 mL, such as at least 0.5 mL, such as at 35 least 0.75 mL, such as at least 1 mL, such as at least 2.5 mL, such as at least 5 mL, such as at least 10 mL, such as at least 20 mL, such as at least 25 mL, such as at least 50 mL, or more.

The containers may be reaction tubes, cuvettes or bioreactors. Such containers are well-known in the art. Preferably, the containers are made of such a material that any reaction performed therein is not affected by it, and which does not affect the optical measurements to be performed.

In some embodiments, the containers of the plurality of containers all have the same dimensions.

In some embodiments, each cassette comprises a plurality of containers arranged in one or more rows, where each row can accommodate 3 containers, 4 containers, 6 containers, 8 containers, 12 containers, 24 containers, 48 containers or 72 containers. This provides flexibility to the assembly and allows for example the assembly to match SBS formats and SLAS standards.

The container, or at least part thereof, such as a lateral side thereof, is made of a material allowing optical access to the inner part of the container at least from the side of the container. This ensures that optical access to the reaction mixture held in the container can be obtained at least when the assembly is in the second configuration.

Sealing Elements and Piercing Elements

In some embodiments, it may be desirable to have access to the containers, particularly from a lower and/or an upper end. Accordingly, in some embodiments the containers comprise a sealing element in their lower end and/or in their upper end, said sealing element preferably comprising the female part of a connecting pair consisting of a female part and a male part, said female part being configured to receive said male part to establish a fluid-tight connection via the connecting pair. Such connecting pair may be a pierceable and hermetically resealable membrane, which allows introduction of one or more piercing elements such as a probe or a hollow piercing element such as a needle. Such a probe can thus be used to monitor one or more parameters of the liquid composition or reaction mixture held within the containers. Hollow piercing elements can be used for example to extract samples from the liquid composition or reaction mixture, to introduce additional compounds such as reagents in the liquid composition or reaction mixture, for example a gas or a further liquid composition.

In some embodiments, the containers have an opening in their upper end. In such embodiments, particularly in embodiments where the assembly is to be used in the context of cell cultures, or any other use requiring controlled conditions, in order to prevent contamination, it may be advantageous that the containers can be capped by capping means. Such capping means may comprise or consist of a further pierceable and hermetically resealable membrane, wherein said further pierceable and hermetically resealable membrane allows introduction of one or more further piercing elements such as a probe or a hollow piercing element such as a needle. Such a probe can thus be used to monitor one or more parameters of the liquid composition or reaction mixture such as a liquid or a molten reaction mixture or medium such as a growth medium held within the containers. Hollow piercing elements can be used for example to extract samples from the liquid composition or reaction mixture, to introduce additional compounds such as reagents in the liquid composition or reaction mixture, for example a gas or a further liquid composition.

The capping means can be removed or opened when it is desirable or necessary to gain physical access to the inner part of the containers, for example when a liquid composition such as a liquid or a molten reaction mixture or medium such as a growth medium is to be introduced therein, or when a sample is to be extracted therefrom. The capping means can afterwards be placed on the containers again, for example to prevent contamination by external agents such as undesirable microorganisms. The capping means also may serve the function of preventing the liquid composition from flowing out of the containers when handling the assembly.

The probe can be configured to monitor one or more parameters of the liquid composition held in the containers, said one or more parameters preferably being selected in the group consisting of temperature, pH, analyte concentration, optical density, turbidity, colorimetric parameters and/or fluorescence.

The piercing element and/or the further piercing element can be configured to allow introduction of one or more compounds such as a gas, for example air, oxygen, ammonia, N₂ or CO₂, in the liquid composition. The piercing element and/or the further piercing element can also, or alternatively, be configured to allow introduction of a further liquid composition such as a growth medium or a buffer, in the liquid composition The piercing element and/or the further piercing element can also, or alternatively, be configured to allow a sample of the liquid composition held in the containers to be taken.

In some embodiments, the piercing element is provided as part of the frame. In such embodiments, a plurality of piercing elements may be provided in the lower part of the frames, each of said piercing elements being configured to penetrate one container in its lower end when the assembly holds the cassette holding the containers.

In some embodiments, the further piercing element is provided as part of the frame. In such embodiments, a plurality of further piercing elements is provided in the upper part of the frames, each of said further piercing elements being configured to penetrate one container in its upper end, optionally wherein each of said further piercing elements is configured to penetrate the capping means, when the assembly holds the cassette holding the containers.

In particular embodiments, the piercing elements are hollow piercing elements such as needles configured to allow bubbling of gas from the lower end of the containers.

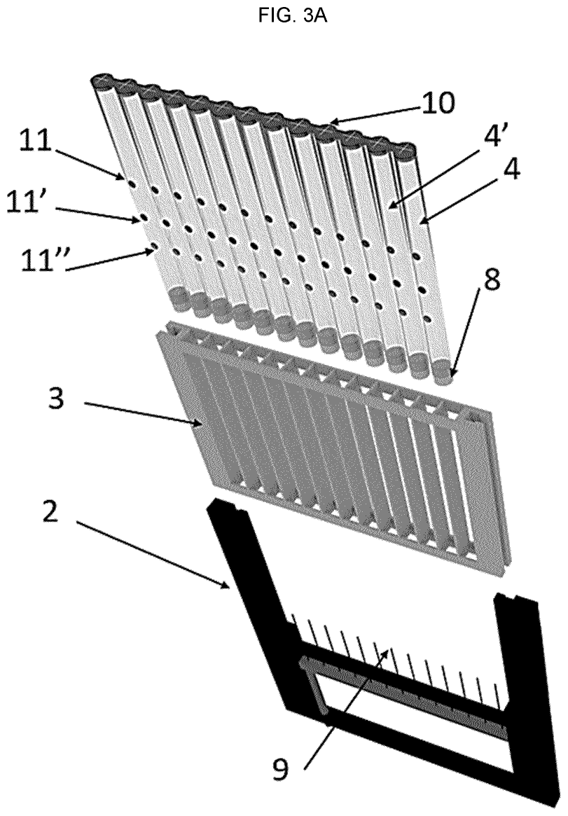

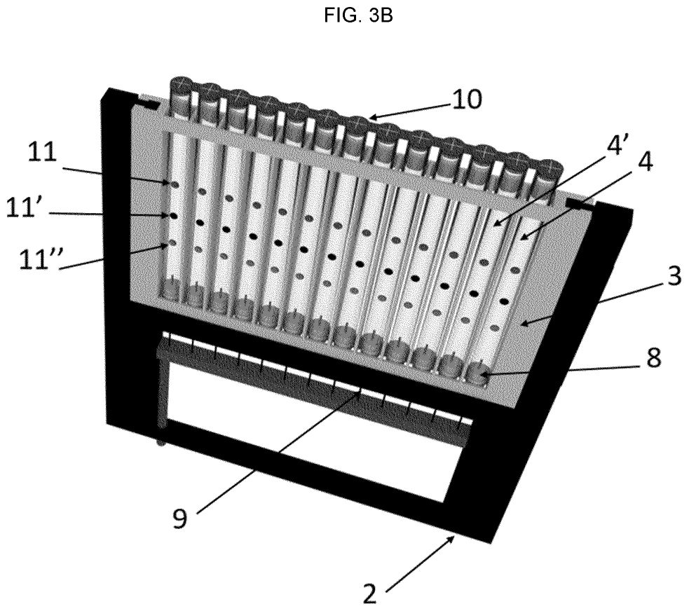

FIGS. 1 and 3 show examples of embodiments where the containers comprise a sealing element in their upper end in the form of a capping strip (10), and where each container further comprises a pierceable and hermetically resalable membrane (8) in their lower end. Further piercing elements (9) can be visualised in those figures, and are here attached to the frame.

Optical Sensors

The containers may comprise one or more optical sensor on their front side, to allow measurement of one or more parameters of the liquid composition such as a liquid or a molten reaction mixture or medium such as a growth medium which is held in the containers in a non-invasive manner, i.e. without having direct, physical access to the liquid composition held within the containers. Such parameters may be CO₂ content, O₂ content, ammonia-N₂ content, pH, analyte concentration, optical density, turbidity, temperature and/or fluorescence. In some embodiments, the optical sensor is an optical spot. This is illustrated by way of example in the figures, in particular FIG. 3 shows a plurality of containers comprising three optical sensors in the form of optical spots (11, 11′, 11″).

Suitable optical sensors and optical spots are readily available to the skilled person. In some such embodiments, a fluorescent indicator dye, sensitive to a given analyte, may be embedded in a coating such as a polymer matrix, which is present on a surface of the containers. Upon light excitation, for example from an LED, the optical sensor is activated. Such optical sensor systems are known from and described in for example EP3168616, WO 2015/019260, DE102014107837, US 13/471,917, U.S. Pat. No. 9,016,573 and WO 02/054045.

In some embodiments, the containers comprise at least one of a first optical sensor configured to measure CO₂ content, a second optical sensor configured to measure O₂ content, and a third optical sensor configured to measure pH. In some embodiments, the containers comprise one of a first optical sensor configured to measure CO₂ content, a second optical sensor configured to measure O₂ content, and a third optical sensor configured to measure pH. In other embodiments, the containers comprise two of a first optical sensor configured to measure CO₂ content, a second optical sensor configured to measure O₂ content, and a third optical sensor configured to measure pH, for example the containers comprise a first optical sensor configured to measure CO₂ content and a second optical sensor configured to measure O₂ content; or the containers comprise a first optical sensor configured to measure CO₂ content and a third optical sensor configured to measure pH; or the containers comprise a second optical sensor configured to measure O₂ content and a third optical sensor configured to measure pH. Additionally, the containers may comprise a fourth optical sensor configured to measure ammonia or content, and/or a fifth optical sensor configured to measure N₂ content, or a fourth optical sensor configured to measure ammonia and N₂ content.

Frame and Cassette Dimensions

It is preferred that the two or more frames of the assembly all have the same dimensions. At any rate, the two or more frames preferably have at least the same height. Optionally, the two or more frames preferably also have the same thickness and/or the same width.

It is also preferred that the two or more cassettes of the assembly all have the same dimensions. At any rate, the two or more cassettes preferably have at least the same height. Optionally, the two or more cassettes preferably also have the same thickness and/or the same width.

Suitable dimensions for the frames and cassettes will be easily determined by the skilled person depending on the desired volume of the reaction system. Some examples of suitable dimensions are provided here. For example, the thickness of each frame and cassette can be between 1 and 25 mm, such as between 2 and 20 mm, such as between 3 and 17.5 mm, such as between 4 and 15 mm, such as between 6 and 12.5 mm, such as between 7 and 10 mm, for example about 8 mm. Each frame and cassette can be between 5 and 50 cm wide, such as between 7.5 and 40 cm wide, such as between 10 and 30 cm wide, such as between 12.5 and 25 cm wide, for example between 13 and 22 cm wide, for example between 15 and 20 cm wide, preferably 13 cm wide. Each frame and cassette can be between 5 and 100 cm high, such as between 7.5 and 90 cm high, for example between 10 and 80 cm high, such as between 12.5 and 75 cm high, for example between 15 and 50 cm high, such as between 18 and 45 cm high, for example between 20 and 30 cm high, such as 25 cm high, 45 cm high or 90 cm high.

In order to allow easy handling of the assembly, the frames and cassettes are preferably configured to match SBS formats and/or SLAS standards as known in the art, at least when the assembly is in the first configuration. Thus in some embodiments, the frames and cassettes are configured to match a 6-tube format, a 12-tube format, a 24-tube format, a 48-tube format, a 96-tube format, a 384-tube format, a 1536-tube format or a 3456-tube format, and even more complex or higher layouts such as a 9600-tube format. As detailed herein above, each cassette can be configured so that the containers are provided in one row or more, such as two rows. The total number of cassettes and frames in one assembly preferably also matches SBS formats and/or SLAS standards.

Thus in some embodiments, the assembly comprises two frames, each comprising one cassette comprising or configured to hold three containers. This matches the 6-tube SBS standard, arranged in 2×3.

In other embodiments, the assembly comprises three frames, each comprising one cassette comprising or configured to hold one row of 4 containers. Alternatively, the assembly comprises four frames, each comprising one cassette comprising or configured to hold one row of 3 containers, or the assembly comprises two frames, each comprising two cassettes comprising or configured to hold two rows of 3 containers. This matches the 12-tube SBS standard, arranged in 3×4.

In other embodiments, the assembly comprises four frames, each comprising one cassette comprising or configured to hold one row of 6 containers, or the assembly comprises two frames, each comprising one cassette comprising or configured to hold two rows of 6 containers. Alternatively, the assembly comprises 6 frames, each comprising one cassette comprising or configured to hold one row of 4 containers, or the assembly comprises three frames, each comprising one cassette comprising or configured to hold two rows of 4 containers. This matches a 24-tube standard, arranged in 4×6.

In the same manner, the assembly comprises cassettes and frames comprising or configured to hold one or two rows of containers to match other SBS standards, such as a 48-tube standard (arranged in 6×8), a 96-tube standard (arranged in 8×12), a 384-tube standard (arranged in 16×24), a 1536-tube standard (arranged in 32×48), a 3456-tube standard (arranged in 48×72) or a 9600-tube format (arranged in 120×80).

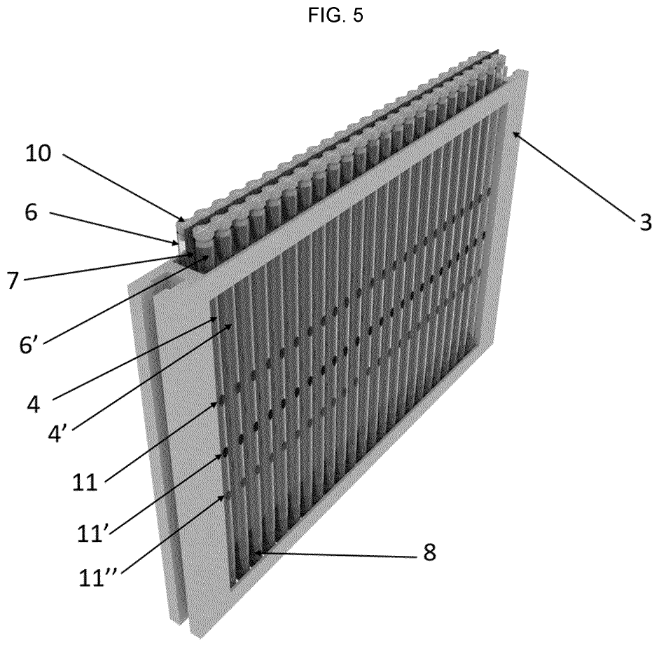

FIG. 5 shows an example of a cassette comprising two rows of containers (6, 6′). In such embodiments, blocking means may be provided, which separate the first and the second row of containers. By way of example, in FIG. 5 the first row of containers (6) is separated from the second row of containers (6′) by a non-transparent barrier (7). The barrier may be in the form of a sheet or a membrane. This allows optical access to only one row of containers at least when the assembly is in the second configuration. When two rows of containers are present within one cassette, the presence of such blocking means thus allows monitoring of the liquid composition within the containers for both rows simultaneously. The cassette can in such cases be flanked by two detection means, which independently each monitor a single row of containers.

Such blocking means may be external to the containers, for example they may comprise or consist of a non-transparent sheet separating said first and second row of containers, or they may be integrated in the containers, for example the blocking means comprise or consist of a coating on the back part of the containers, wherein said coating is configured to prevent optical access to one of said rows, at least when the assembly is in the second configuration.

In embodiments where the blocking means are external to the containers, they may be provided as an integrated part of the cassette, for example they may be formed by mould injection as known in the art. In such embodiments, the blocking means are preferably manufactured from the same material as the cassette.

The blocking means may be made of the same material as the cassettes and/or as the frames, or may be made of plastic, glass or metal, provided that the material is non-transparent and prevents optical access. The skilled person will know which materials are suitable.

The blocking means preferably have such shape and dimensions that optical access to one of said first and second row of containers is enabled from one side of said one row while optical access to the other of said first and second row of containers from said one side of said one row is prevented, at least when the assembly is in the second configuration.

Liquid Composition

The containers of the assembly are configured for receiving and/or holding a liquid composition. In particular, reaction mixtures can be introduced in the containers, for example the containers can be used to run small-scale cultivations or fermentations, and can act as bioreactors. The containers can also be used to run other types of assays, for example enzymatic assays. The term “liquid composition” will herein refer to a liquid composition held in the containers, which may be an inert composition, i.e. a composition in which no reaction takes place, or a reaction mixture, which herein refers to a liquid composition in which a reaction is occurring or at least expected to occur. The reaction mixture may be in a liquid form, for example may be a liquid reaction mixture suitable for a desired assay, or a liquid growth medium in which cells can propagate, or it may be in a molten form, for example it may be a molten reaction mixture or a molten growth medium, such as comprising agar, which is in a liquid form when introduced in the container, but which may subsequently solidify. Such molten reaction mixtures or molten growth media may be suitable if it is desirable to determine parameters such as invasiveness in a solid medium. The liquid composition thus only needs be liquid while it is being introduced in the containers, but may subsequently solidify and become a solid composition, reaction mixture or growth medium.

A reaction mixture (and hence the liquid composition) such as a liquid or a molten reaction mixture or medium such as a growth medium thus may comprise or consist of samples, such as colorimetric samples or biological samples, such as samples comprising cells, tissues, particles and/or enzymes, to be monitored. In other embodiments, the reaction mixture (and hence the liquid composition) such as a liquid or a molten reaction mixture or medium such as a growth medium may comprise or consist of a catalytic system such as an enzymatic system, i.e. a mixture in which an enzyme is present and wherein an enzymatic reaction occurs or is expected to occur, or it may comprise or consist of a cultivation system or a fermentation system, i.e. a mixture in which cells are present, such as microbial cells, prokaryotic cells such as bacterial cells, eukaryotic cells such as yeast cells, fungal cells, insect cells, bird cells, fish cells, or mammalian cells, for example human cells or Chinese Hamster Ovary cells. The cells may be any cell line routinely used in laboratory setups.

System

The present assemblies may be part of a system, which is also provided herein. Herein is thus provided a system comprising:
i) an assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end and wherein:
a) each frame further comprises a hinge (5) or an internal slidable connection, wherein the at two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
      and wherein each frame and cassette are configured to allow optical access to the containers from the side of said each frame and cassette at least when the assembly is in the second configuration,
ii) one or more detection means comprising at least one optical detection means; and
iii) optionally, analysis means.

The system thus comprises any assembly as described in detail herein above, and further comprises one or more detection means which include at least one optical detection means. Suitable detection means include a pH-meter, a thermometer and/or a scale.

In some embodiments, the system comprises an assembly (1) comprising two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end

and wherein each frame further comprises a hinge (5) or an internal slidable connection, wherein the two or more frames are pivotably hinged to one another via said hinge or wherein the two or more frames are connected to one another via said internal slidable connection,

such that in a first configuration the two or more frames abut each other in a longitudinal direction, and in a second configuration the two or more frames are in extension of each other,

and wherein each frame and cassette are configured to allow optical access to the containers from the side of said each frame and cassette at least when the assembly is in the second configuration.

In some embodiments, the system comprises an assembly (1) comprising two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end,

and wherein the assembly comprises a further slidable connection (12, 12′), wherein the two or more frames are releasably connected to one another via said further slidable connection,

such that in a first configuration the two or more frames abut each other in a longitudinal direction, and in a second configuration the two or more frames do not abut each other in a longitudinal direction while still connected to one another via said further slidable connection,

and wherein each frame and cassette are configured to allow optical access to the containers from the side of said each frame and cassette at least when the assembly is in the second configuration.

In some embodiments, the system comprises an assembly (1) comprising two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end,

and wherein each frame further comprises a hinge (5) or an internal slidable connection, wherein the two or more frames are pivotably hinged to one another via said hinge or wherein the two or more frames are connected to one another via said internal slidable connection,

such that in a first configuration the two or more frames abut each other in a longitudinal direction, and in a second configuration the two or more frames are in extension of each other, and wherein the assembly comprises a further slidable connection (12, 12′), wherein the two or more frames are releasably connected to one another via said further slidable connection,

such that in a first configuration the two or more frames abut each other in a longitudinal direction, and in a second configuration the two or more frames do not abut each other in a longitudinal direction while still connected to one another via said further slidable connection,

and wherein each frame and cassette are configured to allow optical access to the containers from the side of said each frame and cassette at least when the assembly is in the second configuration.

Optical detection means may be one or more cameras, a turbidometer, a fluorescence reader, an optical meter or a spectrophotometer, which may be configured to allow optical measurements at least from a lateral side of the assembly when in the second configuration. Such optical detection means may alternatively or additionally be optical sensors as described herein above, for example the containers comprise one or more optical spots on a lateral side. The optical sensors, or parts thereof, can be provided as part of the containers, as described in the context of optical spots. The optical sensors can however also be any other optical detection means capable of measuring an optical parameter from a lateral side of the container. Thus, unlike many systems of the prior art, optical measurements are performed from a lateral side of the assembly. In some embodiments, at least one of the optical detection means is configured to allow optical measurements at least from a lateral side of the assembly when in the second configuration. The additional optical detection means, when present, may be configured to allow optical measurements at least from a lateral side of the assembly, or from a lower side of the assembly, or from an upper side of the assembly.

In some embodiments, the system further comprises an incubation chamber. The incubation chamber is such that it preferably allows incubation of the assembly and of the containers comprised therein in such a manner that incubation can be controlled, and adjusted if necessary. The incubation chamber thus allows control of different parameters which are important for a reaction to be monitored to take place. For example, in the case of an assembly comprising containers in which the reaction systems are cultivation systems or fermentation systems comprising cells, the incubator chamber provides an environment suitable for growth of said cells. For example, temperature and/or atmosphere and/or illumination can be controlled.

The incubator chamber can be equipped with stirring means, for example it can comprise a support in which the assembly can be placed and optionally fastened and maintained in place, where said support can be agitated as is known in the art, in order to homogenise the reaction systems and ensure homogenous distribution of the cells and nutrients and gas such as CO₂, O₂ and/or N₂, in the containers. Such stirring means may be useful also to homogenise the reaction systems prior to performing measurements, in particular optical measurements.

The incubator chamber may be further equipped with feeding containers, which allow e.g. continuous, semi-continuous or punctual introduction of additional liquid compositions, for example fresh cultivation medium or compositions comprising further compounds such as inhibitors or target compounds, in the containers. For instance, the incubator chamber comprises feeding containers, optionally connected to a pump, which can be connected to the piercing elements and/or further piercing elements described herein above. Such feeding containers and optionally pumps can be activated when is necessary or desirable as is known in the art.

The incubator chamber may be further equipped with sampling means, which allow for samples to be collected from the containers at desired times. Such sampling means can be connected to a pump, and can be activated when necessary or desirable as is known in the art.

The incubation chamber may further comprise analysis means, or it may be connected to analysis means. Such analysis means may be a computer. They allow processing of the data collected in the incubation chamber and relating to the system. Thus, the different parameters of the liquid compositions and/or reaction mixtures in the containers can be monitored, and adjusted if necessary.

The incubation chamber preferably has dimensions such that it can accommodate, i.e. receive, the assembly, particularly when the assembly is unfolded (in the second configuration).

The incubation chamber may be further equipped with illumination means, such as a lamp, which allow illumination of the containers when the assembly is in the second configuration, with light of one or more wavelengths. This can be particularly desirable when the reaction systems provide a readout which is dependent on a certain type of light. In some embodiments, the incubation chamber is a dark chamber when the illumination means are turned off, i.e. the assembly can be illuminated in a controlled manner.

The system may further comprise a liquid handler, such as an automated liquid handler or a manual liquid handler. Likewise, particularly when cells are to be introduced in the containers, the system may comprise a colony picker or a replicator such as a cryo-replicator. Preferably the liquid handler, the colony picker and/or the replicator also match an SBS format or an SLAS standard; it will be evident that preferably the liquid handler, the colony picker and/or the replicator match the same SBS format or SLAS standard as the assembly.

FIG. 7 shows an example of such a system, here also comprising an incubation chamber (13) in which the assembly is placed, where the system further comprises optical detection means in the form of one detections means for the non-invasive optical sensor spots (14). In FIG. 6, two optical detection means, here detections means for the non-invasive optical sensor spots (14, 14′), are represented, which in this embodiment allow simultaneous monitoring of a cassette comprising two rows of containers when the assembly is in the second configuration.

Method for Monitoring a Plurality of Reaction Systems and/or for Cultivating and/or Screening a Plurality of Cells

The assembly disclosed herein is particularly advantageous for simultaneous monitoring of a plurality of reaction systems. It can thus be used for example, but not only, to monitor a population of candidate cells for production of compounds of interest, such as new compounds. The assembly can be handled in such a way that the reaction parameters in the reaction systems, i.e. in the containers, can be controlled and monitored. The assembly enables monitoring and/or control of a great number of reaction systems simultaneously, and can easily be scaled up to fit the needs of the user.

Herein is provided a method for monitoring a plurality of reaction systems, said method comprising the steps of:
i) providing a system as disclosed herein or an assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end and wherein:
a) each frame further comprises a hinge (5) or an internal slidable connection, wherein the at two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
      and wherein each frame and cassette are configured to allow optical access to the containers from the side of said each frame and cassette at least when the assembly is in the second configuration,

wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical sensors as described herein,
   thereby monitoring the plurality of reaction systems.

Also provided is a method for cultivating and/or screening a plurality of cells, said method comprising the steps of:
i) providing a system as disclosed herein or an assembly (1) comprising two or more frames (2, 2′) and two or more cassettes (3, 3′), wherein each cassette comprises a plurality of containers or is configured to hold a plurality of containers (4, 4′) in a vertical direction, wherein each frame is configured to receive at least one cassette, wherein each frame comprises an opening in an upper end and wherein:
a) each frame further comprises a hinge (5) or an internal slidable connection, wherein the two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
      and wherein each frame and cassette are configured to allow optical access to the containers from the side of said each frame and cassette at least when the assembly is in the second configuration,
wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical sensors as defined herein,
   the method further comprising introducing said cells in the containers, thereby cultivating and/or screening the plurality of cells.

In the above methods, each individual element may be as described herein above. The assembly may also comprise further elements such as also described herein above.

In a first step of the present methods, an assembly is provided, where the assembly is an assembly as described in detail herein above.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end

and wherein each frame further comprises a hinge (5) or an internal slidable connection, wherein the two or more frames are pivotably hinged to one another via said hinge or wherein the two or more frames are connected to one another via said internal slidable connection,

such that in a first configuration the two or more frames abut each other in a longitudinal direction, and in a second configuration the two or more frames are in extension of each other,

and wherein each frame and cassette are configured to allow optical access to the containers from the side of said each frame and cassette at least when the assembly is in the second configuration.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end, and wherein the assembly comprises a further slidable connection (12, 12′), wherein the two or more frames are releasably connected to one another via said further slidable connection,

such that in a first configuration the two or more frames abut each other in a longitudinal direction, and in a second configuration the two or more frames do not abut each other in a longitudinal direction while still connected to one another via said further slidable connection,

and wherein each frame and cassette are configured to allow optical access to the containers from the side of said each frame and cassette at least when the assembly is in the second configuration.

In some embodiments, the assembly (1) comprises two or more frames (2, 2′) and two or more cassettes (3, 3′), wherein each cassette comprises a plurality of containers or is configured to hold a plurality of containers (4, 4′) in a vertical direction,

wherein each frame is configured to receive at least one cassette,

wherein each frame comprises an opening in an upper end,

and wherein each frame further comprises a hinge (5) or an internal slidable connection, wherein the two or more frames are pivotably hinged to one another via said hinge or wherein the two or more frames are connected to one another via said internal slidable connection,

such that in a first configuration the two or more frames abut each other in a longitudinal direction, and in a second configuration the two or more frames are in extension of each other,

and wherein the assembly comprises a further slidable connection (12, 12′), wherein the two or more frames are releasably connected to one another via said further slidable connection,

such that in a first configuration the two or more frames abut each other in a longitudinal direction, and in a second configuration the two or more frames do not abut each other in a longitudinal direction while still connected to one another via said further slidable connection,

and wherein each frame and cassette are configured to allow optical access to the containers from the side of said each frame and cassette at least when the assembly is in the second configuration.

If the assembly is not in a first configuration, where the two or more frames of the assembly abut each other, the assembly is in a next step arranged in said first configuration. This is illustrated for example in FIG. 1, which shows an assembly comprising frames connected to one another by hinges (5).

The first configuration is well suited for performing the next step, which is the introduction of a liquid composition, such as a liquid or a molten reaction mixture or medium such as a growth medium, in the containers comprised within the assembly. The liquid composition and the reaction mixture may be any of the liquid compositions and reaction mixtures described herein above, such as a liquid or a molten reaction mixture or medium such as a growth medium. In embodiments where the reaction mixture is a molten reaction mixture, this step may further comprise the step of allowing the molten reaction mixture to take a solid form, for example by incubating the container at room temperature or at lower temperatures in order to allow the molten reaction mixture to solidify.

In some embodiments, is introduced in each container a reaction mixture of interest. For example, if it is desirable to cultivate and/or screen a population of cells for production of a compound of interest, different subpopulations can be introduced in each container, where the subpopulation comprises cells of a given genotype, so that all the cells of a given genotype are present in different containers, or the subpopulation may comprise a group of cells having different genotypes, so that in each container several genotypes are represented which form a subgroup of the total genotypes represented within the entire cell population. It will be evident that the cells can also, or alternatively, be all of the same genotype, but may express additional proteins encoded for example by genes introduced in the cell on a vector or plasmid, where the effect of such genes is investigated. In such cases, if it is desirable to cultivate and/or screen a number of genes or genetic elements for their effect on a cell or on the production of a compound of interest, different subpopulations can be introduced in each container, where the subpopulation comprises cells in which a given gene or genetic element has been introduced, so that all the cells expressing a given gene or genetic element are present in different containers. In other cases, the subpopulation may comprise a group of cells expressing a different gene or genetic element, so that in each container several genes or genetic elements are represented, which form a subgroup of the total genes or genetic elements represented within the entire population of genes or genetic elements.

In embodiments where the liquid composition is a molten reaction mixture or a molten growth medium, the cells or reagents may be added before the liquid composition solidifies, at the same time as the liquid composition, or after.

In some embodiments, the liquid composition comprises a reaction mixture, such as a mixture suitable for performing a reaction assay. For example, the liquid composition may be a reaction mixture suitable for performing colorimetric assays. The liquid composition may also comprise a biological sample. The liquid composition may thus comprise cells, tissues, particles and/or enzymes. In some embodiments, the reaction mixture comprises or consists of a catalytic system such as an enzymatic system suitable for performing and/or monitoring an enzymatic reaction, and/or it may comprise a cultivation system or a fermentation system. In the case of cultivation or fermentation systems, preferably the liquid composition is such that it comprises a cell and a suitable incubation medium in which the cell is capable of growing. Such liquid compositions are well known in the art, and include molten compositions such as comprising agar.

In some embodiments, particularly where the assembly matches an SBS format or an SLAS standard as described in detail herein above, the liquid composition can easily be introduced in the containers using an automated liquid handler. Likewise, particularly when cells are to be introduced in the containers, a colony picker or a replicator such as a cryo-replicator can be used. Preferably the liquid handler, the colony picker and/or the replicator also match an SBS format or an SLAS standard; it will be evident that preferably the liquid handler, the colony picker and/or the replicator match the same SBS format or SLAS standard as the assembly.

In a next step, the liquid composition is incubated under conditions allowing the reaction to be monitored to occur in the containers. This can be achieved by placing the assembly in an incubation chamber (13), as exemplified in FIGS. 6 and 7. The assembly can be placed in the incubation system in its first configuration, but it may improve the reaction conditions to arrange the assembly in the second configuration prior to placing it in the incubation chamber. The incubation chamber preferably provides a controlled environment, in which parameters such as temperature,

atmosphere and/or illumination can be adjusted in order to allow a reaction to occur in the reaction mixtures.

The incubation step is performed for a desired duration, preferably for a duration sufficient for the reaction to occur at least partially. The actual duration may vary greatly depending on the type of reactions to be monitored. In the case of enzymatic reactions, some seconds or minutes may be sufficient. In the case of cultivation systems or fermentation systems, the incubation step will generally be longer, for example several hours, days or weeks. The skilled person will have no difficulty in identifying the duration of the incubation step.

If the assembly is not already in the second configuration, the assembly is then arranged in the second configuration. This may require, in embodiments where the assembly is placed in an incubation chamber, in particular in an incubation chamber of small size, that the assembly first be removed from the incubation chamber if already placed therein.

While placing the assembly in a second configuration may facilitate controlling the reaction parameters, it will be understood that the step of incubating the liquid composition under conditions allowing a reaction to occur in the containers and the step of arranging the assembly in the second configuration can occur in any order. In some embodiments, the assembly is placed in the second configuration after the incubation step; in other embodiments, the assembly is placed in the second configuration before the incubation step. The incubation step can also be performed with an assembly being arranged in an intermediate configuration.

In a next step, the reaction in each container is monitored by the means of one or more detection means, comprising at least one optical detection means, as described herein above. Suitable detection means include a pH-meter, a thermometer and/or a scale.

Optical detection means may be one or more cameras, a turbidometer, a fluorescence reader, an optical meter or a spectrophotometer, which may be configured to allow optical measurements at least from a lateral side of the assembly when in the second configuration. Such optical detection means may alternatively or additionally be optical sensors as described herein above, for example the containers comprise one or more optical spots on a lateral side. The optical sensors, or parts thereof, can be provided as part of the containers, as described in the context of optical spots. The optical sensors can however also be any other optical detection means capable of measuring an optical parameter from a lateral side of the container. Thus, unlike many systems of the prior art, optical measurements are in the present methods performed from a lateral side of the assembly. In some embodiments, at least one of the optical detection means is configured to allow optical measurements at least from a lateral side of the assembly when in the second configuration. The additional optical detection means, when present, may be configured to allow optical measurements at least from a lateral side of the assembly, or from a lower side of the assembly, or from an upper side of the assembly.

The detection means may thus allow monitoring of parameters of the liquid composition or reaction mixture such as a liquid or a molten reaction mixture or medium such as a growth medium which is held within the containers. The detection means may comprise the pierceable element(s) and/or the further pierceable element(s) described herein above. The detection means may be a probe such as a sensor configured to measure one or more parameters of the liquid composition held in the containers, said one or more parameters preferably being selected in the group consisting of temperature, pH, analyte concentration, optical density, turbidity, and/or fluorescence.

The detection means and/or the optical detection means may be connected to analysis means, which may facilitate data collection and processing.

It is also possible to measure reaction parameters on samples extracted from the liquid composition held in the containers. Thus in some embodiments, step vi) of the methods further comprises taking a sample of the liquid composition. The liquid composition may, in embodiments where it is a molten composition, in the meantime have assumed a solid form. This can be done by taking advantage of the pierceable element and/or of the further pierceable elements. In such embodiments, step vi) may additionally comprise a step of analysing the sample, for example using analysis means as described herein or as known in the art.

The methods may further comprise a step vii), in which the reaction parameters, in particular the reaction parameters determined in the previous steps, may be adjusted.

For example, fresh cultivation medium or fresh reagents may be introduced in the liquid composition in the containers.

The assemblies and the systems described herein can all be used in any of the above methods.

### EXAMPLES

In the below, particular embodiments of the assembly, system and methods of the present disclosure are described.

FIG. 1 shows an embodiment of an assembly of the present disclosure. In this particular assembly, eight frames (2, 2′) are shown, which can each receive one cassette (3, 3′). The frames comprise here a hinge (5). The assembly is shown in a first configuration, where the frames abut each other in a longitudinal direction. The cassettes can here be slidably inserted in the frames. Each frame comprises a row of needles (9).

The cassettes shown in FIG. 1 can receive each one row of 12 containers (4, 4′) via an opening in their upper end. The containers here have an opening in a lower end and in an upper end. The upper end of the containers is capped by a capping strip (10). The lower end of the containers is capped by individual plugs (8). The capping strip and the individual plugs are pierceable and hermetically resealable. Thus, the needles (9) can pierce the individual plugs, which can however reseal hermetically once the needles are removed.

The containers are shown here with optical sensors in the form of optical spots (11, 11′, 11″). As can be seen, the first configuration here matches a 96-tube SBS format, arranged in 12×8. Liquid or molten compositions can thus easily be introduced in the containers by taking advantage of a matching liquid handler, such as an automated liquid handler.

In another embodiment shown in FIG. 2, the frames (2, 2′) are connected via a slidable connection, here a cylinder (12) on which the frames can slide. The frames each receive one cassette (3, 3′). The assembly is shown in a first configuration, where the frames (2, 2′) abut each other in a longitudinal direction. In this figure, the needles (9) are introduced in the individual plugs (8) at the lower end of the containers (4, 4′). Each frame is here slidable on the cylinder (12), so that the assembly can adopt a second configuration, where the frames (2, 2′) no longer all abut each other in a longitudinal direction, but are still connected via the cylinder (12).

Both embodiments are shown here matching a 96-tube SLS format, arranged in 8×12.

FIG. 3 shows details of a frame (2) equipped with needles (9), where the frame can receive a cassette (3) from an upper end, which cassette can accommodate one row of 12 containers (4, 4′), equipped with a stripping cap (10), individual plugs (8) and three different optical spots (11, 11′, 11″) in exploded view (FIG. 3A) and in assembled view (FIG. 3B). The containers can be used as bioreactors or bubble columns. The needles allow delivery of compounds such as gas, for example air.

FIG. 4 shows the assembly of FIG. 1 , here in an intermediate configuration, which is obtained via the means of the hinge (5). Here the frames are also equipped with magnets (15, 15′), which help maintain the assembly when in the first configuration.

FIG. 5 shows an embodiment of a cassette arrangement which can be used to provide an assembly matching a 384-tube SBS format. Here each cassette can accommodate two rows of 24 containers each, a first row (6) and a second row (6′). The two rows are separated by blocking means, here a non-transparent barrier (7). This allows optical access from one side of the cassette, also when inserted in a frame (not shown) to obtain optical readings from one row of containers, while avoiding that the optical readings are disturbed by the second row of containers, at least when the assembly is in the second configuration. Such a frame can be used to allow simultaneous monitoring of both rows, by providing detection means, in particular optical detection means, on both sides of the frame when the assembly is in the second configuration. When eight such cassettes are inserted in an assembly comprising eight frames, an assembly is thus obtained which can accommodate 384 containers and which matches the 384-SBS standard.

FIG. 6 shows an example of an assembly (1) as described in FIG. 2, here placed in an incubator chamber (13). When arranged in the second configuration, as shown, where not all frames abut each other in a longitudinal direction, optical access to the side of the containers is enabled, and the reaction mixtures comprised in the containers can be monitored for example via detections means for the non-invasive optical sensor spots (14, 14′).

FIG. 7 shows an example of an assembly (1) as described in FIG. 1, here placed in an incubator chamber (13), and arranged in the second configuration. In this particular case, the second configuration is a closed perimeter, and the optical detection means, here a detections means for the non-invasive optical sensor spots (14) has optical access to the side of the containers, thereby enabling monitoring.

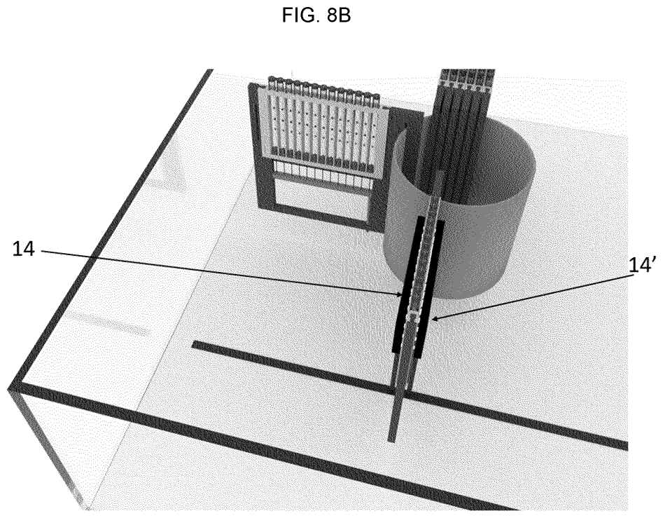

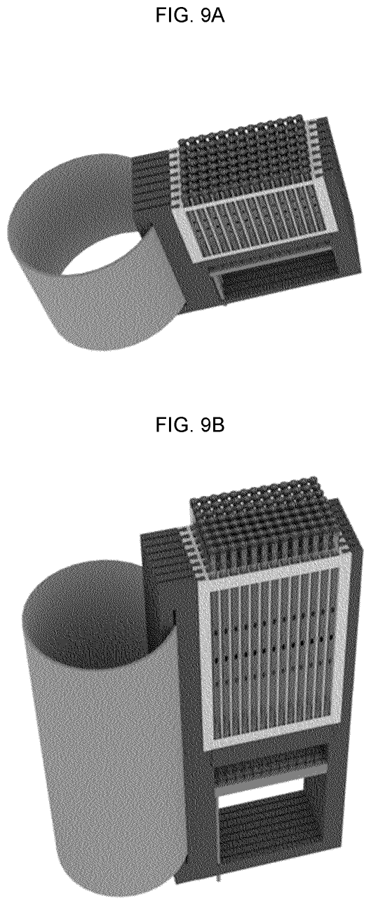

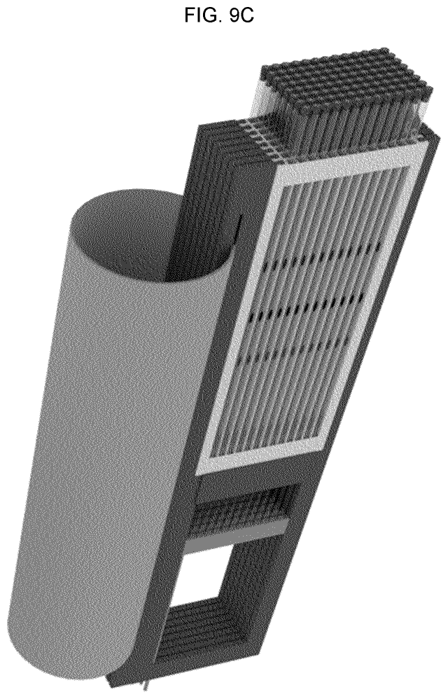

FIG. 8 shows how optical access can be obtained from both sides of a row of containers placed in a cassette placed in a frame. Only one frame and cassette are shown, but the frame is still connected to the remaining frames in this configuration, either directly or indirectly. Here, two detections means for the non-invasive optical sensor spots (14, 14′) are shown. FIG. 8A shows the assembly ready for optical measurements to be performed. FIG. 8B shows the assembly while optical measurements by the detection means (14, 14′) are ongoing.

FIG. 9 shows the assembly of FIG. 2, in three different dimensions. As can be seen, the assemblies and systems of the present disclosure can easily be scaled up by increasing the vertical dimension of the frames, cassettes and containers.

### REFERENCES

EP3168616

WO 2015/019260

DE102014107837

U.S. Pat. No. 13/471,917

U.S. Pat. No. 9,016,573

WO 02/054045

U.S. Pat. No. 7,422,893

WO 2020/079211

Items
1. An assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end and wherein:
      a) each frame further comprises a hinge (5) or an internal slidable connection, wherein the two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
      b) the assembly comprises a further slidable connection (12, 12′), wherein the two or more frames are releasably connected to one another via said further slidable connection, such that in a first configuration the two or more frames abut each other in a longitudinal direction, and in a second configuration the two or more frames do not abut each other in a longitudinal direction while still connected to one another via said further slidable connection,
         and wherein each frame and cassette are configured to allow optical access to the containers from the side of said each frame and cassette at least when the assembly is in the second configuration.
2. An assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end
   and wherein each frame further comprises a hinge (5) or an internal slidable connection, wherein the two or more frames are pivotably hinged to one another via said hinge or wherein the two or more frames are connected to one another via said internal slidable connection,
   such that in a first configuration the two or more frames abut each other in a longitudinal direction, and in a second configuration the two or more frames are in extension of each other,
   and wherein each frame and cassette are configured to allow optical access to the containers from the side of said each frame and cassette at least when the assembly is in the second configuration.
3. An assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end,
   and wherein the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration.
4. An assembly (1) comprising two or more frames (2, 2′) and two or more cassettes (3, 3′), wherein each cassette comprises a plurality of containers or is configured to hold a plurality of containers (4, 4′) in a vertical direction,
   wherein each frame is configured to receive at least one cassette,
   wherein each frame comprises an opening in an upper end,
   and wherein each frame further comprises a hinge (5) or an internal slidable connection, wherein the two or more frames are pivotably hinged to one another via said hinge or wherein the two or more frames are connected to one another via said internal slidable connection,
   such that in a first configuration the two or more frames abut each other in a longitudinal direction, and in a second configuration the two or more frames are in extension of each other,
   and wherein the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration.
5. The assembly according to any one of the preceding items, wherein each frame comprises an internal slidable connection, preferably on a lateral surface, more preferably on an upper portion and/or a lower portion of the lateral surface.
6. The assembly according to item 5, wherein the internal slidable connection is selected from the group consisting of one or more ridges and/or one or more magnets.
7. The assembly according to any one of the preceding items, wherein the assembly comprises a further slidable connection such as a ring or a hollow cylinder to which the two or more frames are attached.
8. The assembly according to any one of the preceding items, wherein the container is a reaction tube, a cuvette or a bioreactor.
9. The assembly according to any one of the preceding items, wherein each container is configured to hold a liquid composition such as a liquid or a molten reaction mixture or medium such as a growth medium.
10. The assembly according to any one of the preceding items, wherein the liquid composition and/or the reaction mixture comprises or consists of samples such as colorimetric samples or biological samples, such as samples comprising cells, tissues, particles and/or enzymes; and/or the liquid composition and/or the reaction mixture comprises or consists of a catalytic system such as an enzymatic system, a cultivation system or a fermentation system.
11. The assembly according to any one of the preceding items, wherein each container is configured to hold a volume of the liquid composition of at least 1 μL, such as at least 2.5 μL, such as at least 5 μL, such as at least 10 μL, such as at least 15 μL, such as at least 25 μL, such as at least 50 μL, such as at least 75 μL, such as at least 0.1 mL, such as at least 0.25 mL, such as at least 0.5 mL, such as at least 0.75 mL, such as at least 1 mL, such as at least 2.5 mL, such as at least 5 mL, such as at least 10 mL, such as at least 20 mL, such as at least 25 mL, such as at least 50 mL, or more.
12. The assembly according to any one of the preceding items, wherein each container, or at least a part thereof, is made of a material allowing optical access to the inner part of the container at least from the side of the container.
13. The assembly according to any one of the preceding items, wherein the two or more frames all have the same dimensions.
14. The assembly according to any one of the preceding items, wherein the two or more cassettes all have the same dimensions.
15. The assembly according to any one of the preceding items, wherein the containers all have the same dimensions.
16. The assembly according to any one of the preceding items, wherein each cassette and/or frame is configured to match a standard SBS format and/or an SLAS standard when the assembly is in the first configuration, such as a 6-tube format, a 12-tube format, a 24-tube format, a 48-tube format, a 96-tube format, a 384-tube format, a 1536-tube format, a 3456-tube format or a 9600-tube format.
17. The assembly according to any one of the preceding items, wherein the plurality of containers is provided in one or more row, such as at least two rows, and wherein each cassette is configured to receive and to hold said one or more row, such as said at least two rows.
18. The assembly according to any one of the preceding items, wherein the one or more row is a first row (6) and a second row (6′) of containers.
19. The assembly according to any one of the preceding items, further comprising blocking means (7) separating said first and second row of containers.
20. The assembly according to any one of the preceding items, wherein the blocking means comprise or consist of a non-transparent barrier such as a non-transparent sheet or membrane separating said first and second row of containers, and/or wherein the blocking means comprise or consist of a coating on the back part of the containers, wherein said coating is configured to prevent optical access to one of said rows.
21. The assembly according to any one of the preceding items, wherein the non-transparent sheet and /or the coating is of dimensions such that optical access to one of said first and second row of containers is enabled from one side of said one row while optical access to the other of said first and second row of containers from said one side of said one row is prevented.
22. The assembly according to any one of the preceding items, wherein the two or more frames can receive the two or more cassettes from an upper end.
23. The assembly according to any one of the preceding items, wherein the two or more frames comprise an inner cavity on a lateral surface, or wherein the two or more frames comprise a transparent lateral surface, thereby allowing optical access to the cassette held in the assembly at least when the assembly is in the second configuration.
24. The assembly according to any one of the preceding items, wherein the cassettes comprise openings on their lateral side, or wherein the cassettes are made of a material allowing optical access through their lateral side, thereby enabling optical access to the containers held therein at least when the assembly is in the second configuration.
25. The assembly according to any one of the preceding items, wherein the inner cavity of the two or more frames and/or the openings on the lateral side of the cassettes consists of a material which allows transmission of light, such as UV light, laser light or blue light.
26. The assembly according to any one of the preceding items, wherein the containers are provided with a sealing element in their lower end, said sealing element preferably comprising the female part of a connecting pair consisting of a female part and a male part, said female part being configured to receive said male part to establish a fluid-tight connection via the connecting pair.
27. The assembly according to any one of the preceding items, wherein the containers are provided with a pierceable and hermetically resealable membrane (8) at least in their lower end, wherein said pierceable and hermetically resealable membrane allows introduction of one or more piercing elements (9) such as a probe or a hollow piercing element such as a needle.
28. The assembly according to any one of the preceding items, wherein the containers have an opening in their upper end, optionally wherein the opening is capped by capping means (10).
29. The assembly according to any one of the preceding items, wherein the capping means comprises or consists of a further pierceable and hermetically resealable membrane, wherein said further pierceable and hermetically resealable membrane allows introduction of one or more further piercing elements such as a probe or a hollow piercing element such as a needle.
30. The assembly according to any one of the preceding items, wherein the piercing element and/or the further piercing element is a probe such as a sensor configured to measure one or more parameters of the liquid composition held in the containers, said one or more parameters preferably being selected in the group consisting of temperature, pH, analyte concentration, optical density, turbidity, colorimetric parameters and/or fluorescence.
31. The assembly according to any one of the preceding items, wherein the piercing element and/or the further piercing element is a needle configured to allow introduction of one or more compounds such as a gas, for example air, oxygen, ammonia, N₂ or CO₂, and/or of a further liquid composition such as a growth medium or a buffer, in the liquid composition, and/or configured to allow a sample of the liquid composition held in the containers to be taken.
32. The assembly according to any one of the preceding items, wherein a plurality of piercing elements is provided in the lower part of the frames, each of said piercing elements being configured to penetrate one container in its lower end when the assembly holds the cassette holding the containers.
33. The assembly according to any one of the preceding items, wherein a plurality of further piercing elements is provided in the upper part of the frames, each of said further piercing elements being configured to penetrate one container in its upper end, optionally wherein each of said further piercing elements is configured to penetrate the capping means, when the assembly holds the cassette holding the containers.
34. The assembly according to any one of the preceding items, wherein the piercing elements are hollow piercing elements configured to allow bubbling of gas from the lower end of the containers.
35. The assembly according to any one of the preceding items, wherein the containers comprise one or more optical sensor (11, 11′, 11″) on their front side, such as an optical spot, said optical sensor being configured to allow measurement of one or more parameters of the liquid composition held in the containers, such as CO₂ content, O₂ content, ammonia content, N₂ content, pH, analyte concentration, optical density, turbidity, temperature and/or fluorescence, preferably the containers comprise each at least one of a first optical sensor configured to measure CO₂ content, a second optical sensor configured to measure O₂ content, and a third optical sensor configured to measure pH.
36. The assembly according to any one of the preceding items, wherein the two frames located in the extreme positions of the assembly when in the first configuration can be connected to one another such that the assembly forms a closed perimeter.
37. The assembly according to any one of the preceding items, wherein the two or more frames further comprise additional connection means such as magnets, configured to facilitate maintaining the assembly in the first configuration.
38. The assembly according to any one of the preceding items, wherein the two or more frames are manufactured of an inert, UV-resistant material such as plastic or metal.
39. A system comprising:
   i) An assembly (1) comprising two or more frames (2, 2′) and two or more cassettes (3, 3′), wherein each cassette comprises a plurality of containers or is configured to hold a plurality of containers (4, 4′) in a vertical direction,
      wherein each frame is configured to receive at least one cassette,
      wherein each frame comprises an opening in an upper end and wherein:
         a) each frame further comprises a hinge (5) or an internal slidable connection, wherein the at two or more frames are pivotably hinged to one another via said hinge or wherein the two or more frames are connected to one another via said internal slidable connection, such that in a first configuration the two or more frames abut each other in a longitudinal direction, and in a second configuration the two or more frames are in extension of each other, and/or
         b) the assembly comprises a further slidable connection (12, 12′), wherein the two or more frames are releasably connected to one another via said further slidable connection, such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration,
   ii) one or more detection means comprising at least one optical detection means (14, 14′); and
   iii) optionally, analysis means.
40. A system comprising:
   i) An assembly according to any one of items 2 or 5 to 38,
   ii) one or more detection means comprising at least one optical detection means (14, 14′); and
   iii) optionally, analysis means.
41. A system comprising:
   i) An assembly according to any one of items 3 or 5 to 38,
   ii) one or more detection means comprising at least one optical detection means (14, 14′); and
   iii) optionally, analysis means.
42. A system comprising:
   i) An assembly according to any one of items 4 to 38,
   ii) one or more detection means comprising at least one optical detection means (14, 14′); and
   iii) optionally, analysis means.
43. The system according to any one of items 39 to 42, wherein the assembly is as defined in any one of items 1 to 38.
44. The system according to any one of items 39 to 43, wherein the detection means is a pH-meter, a thermometer and/or a scale.
45. The system according to any one of items 39 to 44, wherein the optical detection means is as a camera, a turbidometer, a fluorescence reader, an optical meter or a spectrophotometer.
46. The system according to any one of items 39 to 45, wherein at least one of the at least one optical detection means is configured to have optical access to and provide optical measurements from the containers from a lateral side of the assembly.
47. The system according to any one of items 39 to 46, wherein each cassette and/or frame is configured to match a standard SBS format and/or an SLAS standard when the assembly is in the first configuration, such as a 6-tube format, a 12-tube format, a 24-tube format, a 48-tube format, a 96-tube format, a 384-tube format, a 1536-tube format, a 3456-tube format or a 9600-tube format.
48. The system according to any one of items 39 to 47, further comprising an incubation chamber (13).
49. A method for monitoring a plurality of reaction systems, said method comprising the steps of:
   i) providing a system according to any one of items 39 to 48 or an assembly (1) comprising two or more frames (2, 2′) and two or more cassettes (3, 3′), wherein each cassette comprises a plurality of containers or is configured to hold a plurality of containers (4, 4′) in a vertical direction,
      wherein each frame is configured to receive at least one cassette,
      wherein each frame comprises an opening in an upper end and wherein:
   a) each frame further comprises a hinge (5) or an internal slidable connection, wherein the at two or more frames are pivotably hinged to one another via said hinge or wherein the two or more frames are connected to one another via said internal slidable connection,
      such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
   such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration,
   wherein each reaction system comprises or consists of one of said containers (4, 4′),
   preferably wherein the assembly is according to any one of items 1 to 38,
   ii) if the assembly is not in the first configuration, arranging the assembly in the first configuration,
   iii) introducing a liquid composition such as a liquid or a molten reaction mixture or medium such as a growth medium in the containers (4, 4′),
   iv) incubating the liquid composition under conditions allowing a reaction to occur in the containers,
   v) arranging the assembly in the second configuration, and
   vi) monitoring the reaction in the containers by the means of one or more detection
      means comprising at least one optical detection means, such as optical detection means as defined in any one of the preceding items, thereby monitoring the plurality of reaction systems.
50. A method for cultivating and/or screening a plurality of cells, said method comprising the steps of:
   i) providing a system according to any one of items 39 to 48 or an assembly (1) comprising two or more frames (2, 2′) and two or more cassettes (3, 3′), wherein each cassette comprises a plurality of containers or is configured to hold a plurality of containers (4, 4′) in a vertical direction,
      wherein each frame is configured to receive at least one cassette,
      wherein each frame comprises an opening in an upper end and wherein:
   a) each frame further comprises a hinge (5) or an internal slidable connection, wherein the two or more frames are pivotably hinged to one another via said hinge or wherein the two or more frames are connected to one another via said internal slidable connection,
      such that in a first configuration the two or more frames abut each other in a longitudinal direction, and in a second configuration the two or more frames are in extension of each other, and/or
   b) the assembly comprises a further slidable connection (12, 12′), wherein the two or more frames are releasably connected to one another via said further slidable connection,
      such that in a first configuration the two or more frames abut each other in a longitudinal direction, and in a second configuration the two or more frames do not abut each other in a longitudinal direction while still connected to one another via said further slidable connection,
   and wherein each frame and cassette are configured to allow optical access to the containers from the side of said each frame and cassette at least when the assembly is in the second configuration,
      wherein each reaction system comprises or consists of one of said containers (4, 4′),
      preferably wherein the assembly is according to any one of items 1 to 38,
   ii) if the assembly is not in the first configuration, arranging the assembly in the first configuration,
   iii) introducing a liquid composition such as a liquid or a molten reaction mixture or medium such as a growth medium in the containers (4, 4′),
   iv) incubating the liquid composition under conditions allowing a reaction to occur in the containers,
   v) arranging the assembly in the second configuration, and
   vi) monitoring the reaction in the containers by the means of one or more detection means comprising at least one optical detection means, such as optical sensors as defined in any one of the preceding items,
      the method further comprising introducing said cells in the containers before or together with step iii) or iv),
      thereby cultivating and/or screening the plurality of cells.
51. The method according to any one of items 49 to 50, wherein the reaction

system is a cultivation system, a fermentation system or a bioreactor.
52. The method according to any one of items 49 to 51, wherein the optical detection means have optical access to a lateral side of the containers at least when the assembly is in the second configuration.
53. The method according to any one of items 49 to 52, wherein the detection means is a pH-meter, a thermometer, a scale and/or the optical detection means is as a camera, a turbidometer, a fluorescence reader, an optical meter or a spectrophotometer.
54. The method according to any one of items 49 to 53, wherein the containers are provided with a pierceable and hermetically resealable membrane (8) at least in their lower end, wherein said pierceable and hermetically resealable membrane allows introduction of one or more piercing elements (9) such as a probe or a hollow piercing element such as a needle.
55. The method according to any one of items 49 to 54, wherein step iii) and/or step iv) and/or step vi) comprise introducing in the container one or more compounds such as a gas, for example air, oxygen, ammonia, N₂ or CO₂, and/or of a further liquid composition such as a growth medium or a buffer, by introducing said one or more piercing elements into the containers through said pierceable and hermetically resealable membrane.
56. The method according to any one of items 49 to 55, wherein the containers have an opening in their upper end, optionally wherein the opening is capped by capping means (10).
57. The method according to any one of items 49 to 56, wherein step ii) comprises introducing the liquid composition before placing the capping means on the containers.
58. The method according to any one of items 49 to 57, wherein the capping means comprises or consists of a further pierceable and hermetically resealable membrane, wherein said further pierceable and hermetically resealable membrane allows introduction of one or more further piercing elements such as a probe or a hollow piercing element such as a needle, and wherein step ii) comprises introducing the liquid composition by introducing said one or more further piercing elements into the containers through said further pierceable and hermetically resealable membrane.
59. The method according to any one of items 49 to 58, further comprising the step of providing one or more cells in the liquid composition before, after or simultaneously with step iii), preferably in step ii), step iii) and/or step iv).
60. The method according to any one of items 49 to 59, wherein the liquid composition is a growth medium configured to allow growth of said one or more cells.
61. The method according to any one of items 49 to 60, wherein the cell is a bacterial cell or a eukaryotic cell, such as a yeast cell, a fungal cell, an insect cell, a fish cell, a mammalian cell or a plant cell.
62. The method according to any one of items 49 to 61, wherein step ii) comprises or consists of introducing the liquid composition using a liquid dispenser, preferably a liquid dispenser matching a standard SBS format and/or an SLAS standard , such as a 6-tube format, a 12-tube format, a 24-tube format, a 48-tube format, a 96-tube format, a 384-tube format, a 1536-tube format, a 3456-tube format or a 9600-tube format.
63. The method according to any one of items 49 to 62, wherein step ii) comprises or consists of introducing the liquid composition and/or reaction mixture using a liquid handler, such as a manual or automated liquid handler.
64. The method according to any one of items 49 to 63, wherein step ii) comprises or consists of introducing the cells using a colony picker or a replicator, such as a cryo-replicator, preferably matching a standard SBS format and/or an SLAS standard, such as a 6-tube format, a 12-tube format, a 24-tube format, a 48-tube format, a 96-tube format, a 384-tube format, a 1536-tube format, a 3456-tube format or a 9600-tube format.
65. The method according to any one of items 49 to 64, wherein step iii) is performed under conditions allowing growth of said one or more cells.
66. The method according to any one of items 49 to 65, wherein the pierceable element and/or the further pierceable element is a needle, and wherein step vi) comprises taking a sample of the liquid composition through said needle, optionally wherein step vi) further comprises a step of analysing the sample.
67. The method according to any one of items 49 to 66, wherein the pierceable element and/or the further pierceable element is a probe such as a sensor configured to measure one or more parameters of the liquid composition held in the containers, said one or more parameters preferably being selected in the group consisting of temperature, pH, analyte concentration, optical density, turbidity, and/or fluorescence, and wherein step vi) comprises determining one or more parameters of the reaction systems by the means of said probe.
68. The method according to any one of items 49 to 67, wherein the containers comprise one or more optical sensor (11, 11′, 11″) on their front side, such as an optical spot, said optical sensor being configured to allow measurement of one or more parameters of the liquid composition held in the containers, such as CO₂ content, O₂ content, ammonia content, N₂ content, pH, analyte concentration, optical density, turbidity, temperature and/or fluorescence, preferably the containers comprise each at least one of a first optical sensor configured to measure CO₂ content, a second optical sensor configured to measure O₂ content, and a third optical sensor configured to measure pH.
69. The method according to any one of items 49 to 68, wherein detection means, and/or the optical detection means such as the optical sensor and/or the probe are connected to analysis means.
70. The method according to any one of items 49 to 69, further comprising a step vii) comprising or consisting of adjusting the reaction parameters.
71. The method according to any one of items 49 to 70, wherein step iv) is performed in an incubation chamber (13), optionally wherein the incubation chamber is a dark chamber.
72. Use of the assembly according to any one of items 1 to 38 or the system according to any one of items 39 to 47, in a method according to any one of items 49 to 71.

1. An assembly (1) comprising two or more frames (2, 2′) and two or more cassettes (3, 3′), wherein each cassette comprises a plurality of containers or is configured to hold a plurality of containers (4, 4′) in a vertical direction, wherein each frame is configured to receive at least one cassette, wherein each frame comprises an opening in an upper end and wherein:
a) each frame further comprises a hinge (5) or an internal slidable connection, wherein the two or more frames are pivotably hinged to one another via said hinge or wherein the two or more frames are connected to one another via said internal slidable connection,
such that in a first configuration the two or more frames abut each other in a longitudinal direction, and in a second configuration the two or more frames are in extension of each other, and/or
b) the assembly comprises a further slidable connection (12, 12′), wherein the two or more frames are releasably connected to one another via said further slidable connection,
such that in a first configuration the two or more frames abut each other in a longitudinal direction, and in a second configuration the two or more frames do not abut each other in a longitudinal direction while still connected to one another via said further slidable connection,
and wherein each frame and cassette are configured to allow optical access to the containers from the side of said each frame and cassette at least when the assembly is in the second configuration.

2. The assembly according to claim 1, wherein each frame comprises an internal slidable connection, preferably on a lateral surface, more preferably on an upper portion and/or a lower portion of the lateral surface, and/or wherein the assembly comprises a further slidable connection such as a ring or a hollow cylinder to which the two or more frames are attached.

3. The assembly according to any one of the preceding claims, wherein the container is a reaction tube, a cuvette or a bioreactor, and/or wherein each container is configured to hold a liquid composition such as a liquid or a molten reaction mixture or medium such as a growth medium.

4. The assembly according to any one of the preceding claims, wherein each container, or at least a part thereof, is made of a material allowing optical access to the inner part of the container at least from the side of the container.

5. The assembly according to any one of the preceding claims, wherein each cassette and/or frame is configured to match a standard SBS format and/or an SLAS standard when the assembly is in the first configuration, such as a 6-tube format, a 12-tube format, a 24-tube format, a 48-tube format, a 96-tube format, a 384-tube format, a 1536-tube format, a 3456-tube format or a 9600-tube format.

6. The assembly according to any one of the preceding claims, wherein the plurality of containers is provided in one or more row, such as at least two rows, and wherein each cassette is configured to receive and to hold said one or more row, such as said at least two rows, preferably wherein the one or more row is a first row (6) and a second row (6′) of containers, optionally wherein the assembly further comprises blocking means (7) separating said first and second row of containers, preferably wherein the blocking means comprise or consist of a non-transparent barrier such as a non-transparent sheet or membrane separating said first and second row of containers, and/or wherein the blocking means comprise or consist of a coating on the back part of the containers, wherein said coating is configured to prevent optical access to one of said rows, preferably wherein the non-transparent sheet and/or the coating is of dimensions such that optical access to one of said first and second row of containers is enabled from one side of said one row while optical access to the other of said first and second row of containers from said one side of said one row is prevented.

7. The assembly according to any one of the preceding claims, wherein the containers are provided with a sealing element in their lower end, said sealing element preferably comprising the female part of a connecting pair consisting of a female part and a male part, said female part being configured to receive said male part to establish a fluid-tight connection via the connecting pair, and/or wherein the containers have an opening in their upper end, optionally wherein the opening is capped by capping means (10).

8. The assembly according to any one of the preceding claims, wherein a plurality of piercing elements is provided in the lower part of the frames, each of said piercing elements being configured to penetrate one container in its lower end when the assembly holds the cassette holding the containers.

9. The assembly according to any one of the preceding claims, wherein the containers comprise one or more optical sensor (11, 11′, 11″) on their front side, such as an optical spot, said optical sensor being configured to allow measurement of one or more parameters of the liquid composition held in the containers, such as CO₂ content, O₂ content, ammonia content, N₂ content, pH, analyte concentration, optical density, turbidity, temperature and/or fluorescence, preferably the containers comprise each at least one of a first optical sensor configured to measure CO₂ content, a second optical sensor configured to measure O₂ content, and a third optical sensor configured to measure pH.

10. A system comprising:
i) An assembly two or moreaccording to any one of the preceding claims,
ii) one or more detection means comprising at least one optical detection means (14, 14′); and
iii) optionally, analysis means.

11. The system according to claim 10, further comprising an incubation chamber (13).

12. A method for monitoring a plurality of reaction systems, said method comprising the steps of:
i) providing a system according to any one of claims 10 to 11 or an assembly according to any one of claims 1 to 9,
wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical detection means as defined in any one of the preceding claims, preferably wherein the optical detection means have optical access to a lateral side of the containers at least when the assembly is in the second configuration
thereby monitoring the plurality of reaction systems.

13. A method for cultivating and/or screening a plurality of cells, said method comprising the steps of:
i) providing a system according to any one of claims 10 to 11 or an assembly according to any one of claims 1 to 9,
wherein each reaction system comprises or consists of one of said containers (4, 4′),
ii) if the assembly is not in the first configuration, arranging the assembly in the first configuration,
iii) introducing a liquid composition such as a liquid or a molten reaction mixture or medium such as a growth medium in the containers (4, 4′),
iv) incubating the liquid composition under conditions allowing a reaction to occur in the containers,
v) arranging the assembly in the second configuration, and
vi) monitoring the reaction in the containers by the means of one or more detection means comprising at least one optical detection means, such as optical sensors as defined in any one of the preceding claims, the method further comprising introducing said cells in the containers before or together with step iii) or iv),
thereby cultivating and/or screening the plurality of cells.

14. The method according to any one of claims 12 to 13, wherein the optical detection means have optical access to a lateral side of the containers at least when the assembly is in the second configuration.

15. Use of the assembly according to any one of claims 1 to 9 or the system according to any one of claims 10 to 11, in a method according to any one of claims 12 to 14.
